(19) United States
(12) Patent Application Publication
(10) Pub. No.: US 2011/0124069 A1
(43) Pub. Date: May 26, 2011

(54) PRODUCTION METHOD

(76) Inventors: Jorg Mampel, Bensheim (DE); Guido Meurer, Seeheim-Jugenheim (DE); Jurgen Eck, Bensheim (DE)

(21) Appl. No.: 13/003,930

(22) PCT Filed: Jul. 16, 2009
(86) PCT No.: PCT/EP2009/059132

§ 371 Date: Jan. 13, 2011

(30) Foreign Application Priority Data
Jul. 28, 2008 (EP) 08161267.3
Aug. 28, 2008 (EP) 08163201.0

(51) Int. Cl. C12P 7/18; C12N 1/21

(52) U.S. Cl. 435/158; 435/252.33

(57) ABSTRACT

The invention relates to the development of microorganisms that produce 1,2-propanediol (1,2-PD) from glycerol, whereas glycerol is simultaneously the substrate carbon source for 1,2-PD- and biomass production. The invention demonstrates that any type of glycerol serves as carbon substrate for 1,2-PD biosynthesis. The microorganism is a recombinant organism, preferentially an E. coli K12 strain or a derivative thereof, particularly a strain, which is inactivated in competing pathways that lower 1,2-PD production.

The present invention relates to a method for the production of 1,2-propanediol from glycerol in host cells. More specifically the present invention describes recombinant enzymatic activities which enable the synthesis of exclusively the 1,2-isomer of propanediol from pure and crude preparations of glycerol. The present invention also provides suitable combinations of overexpression and inactivation of key-activities for the production of 1,2-propanediol.

1,2-propanediol (propylene glycol; 1,2-PD) is a major bulk chemical that is widely used as a component of unsaturated polyester resins, pharmaceutical formulations and cosmetics, liquid detergents, coolants and anti-freeze or de-icing fluids. Since 1,2-PD is optically active, enantiomerically pure preparations of 1,2-PD might be of special interest for medical, agricultural or physiological applications.

1,2-propanediol is currently produced from petrochemicals by chemical synthesis that involves handling of large amounts of toxic compounds like epichlorhydrin or hydroperoxid. In conventional chemical synthesis, 1,2-PD is obtained by the hydration of propylene oxide, which is produced from propylene. The chemical synthesis yields racemic 1,2-PD and demands large amounts of water in order to prevent formation of polyglycol. Conventional chemical synthesis is dependent on fossil resources and leads to the production of large amounts of by-products; thus it appears problematic in terms of environmental and economical aspects.

It is known that 1,2 propanediol can be produced by microorganisms from sugars as substrates (Kluyver and Schellen, 1937) (Heath, E. C., 1962) (Altaras, N. E, 2001) (Tran Din, K, 198) (Cameron, D. C., 1986) (Cameron, D. C, 1998) (Park, Y. H., 2006; U.S. Pat. No. 7,049,109 B2).

U.S. Pat. No. 6,087,140 and U.S. Pat. No. 6,303,352 describe the production of 1,2-PD from sugars except 6-deoxyhexoses by recombinant organisms.

In WO 2005/073364, US 2007/072279 a method is described by which microorganisms are generated and selected that show enhanced capabilities to produce 1,2-PD from unspecified carbon sources. More specifically, inactivation of a set of genes is taught to create strains harbouring single or multiple mutations that are the basis for a subsequent selection procedure by chemostat-fermentations. The focus of the application is on the inactivation of the genes encoding an aldA and gloA activity. All disclosed examples are given for E. coli MG1655 that has mutations in at least the following two genes: triosephosphat-isomerase (tpiA) and both subunits of pyruvate-formate lyase (pflAB). Furthermore, the examples specifically refer to glucose as carbon-source for fermentations.

There is therefore an unmet need in the art for improving the biotechnological processes for the production of 1,2-propanediol (1,2-PD). There is further an unmet need for improved microbial strains that can be used in such a process.

The present invention addresses this unmet need by providing solutions to the problems that had so far prevented significant improvements in this area.

To solve these problems, the present invention provides an improved biotechnological process for the production of 1,2 propanediol (1,2-PD) from a non-fermentable, inexpensive carbon substrate, whereby the carbon substrate is sustaining production of biomass and serves as a substrate for production of 1,2 propanediol (1,2-PD) at the same time. The present invention further provides improved microbial strains which are specifically adapted to the specific requirements of this procedure and are therefore specifically suited for use in the process according to the invention.

In particular, the present invention provides a host cell, particularly a microorganism or strain, which is engineered to produce high levels of 1,2 propanediol (1,2-PD) when grown on a non-fermentable carbon substrate, whereby the carbon substrate is sustaining production of biomass and serves as a substrate for production of 1,2 propanediol (1,2-PD) at the same time, particularly when grown on glycerol as the sole carbon source.

In one embodiment of the invention, a host cell, particularly a microorganism or strain, is provided which is engineered to produce high levels of 1,2 propanediol (1,2-PD) when grown on glycerol as the sole carbon source, wherein said glycerol has a degree of purity of at least 70%, particularly of at least 75%, particularly of at least 80%, particularly of at least 85%, particularly of at least 90%, particularly of at least 95%, particularly of at least 99% and up to 100%, with all integers falling within the above defined ranges also being comprised herewith.

In a specific embodiment, the glycerol has a degree of purity of between 80% and 90%, particularly of about 85%.

In one embodiment of the invention, a host cell, particularly a microorganism or strain, is provided which is capable of producing high levels of 1,2 propanediol (1,2-PD) when grown on glycerol as the sole carbon source, wherein said host cell, particularly said microorganism or strain, is engineered to overexpress propanediol oxidoreductase (fucO), particularly by introducing a gene encoding a propanediol oxidoreductase (fucO) activity.

In one embodiment, the invention provides a host cell, particularly a microorganism or strain, which is capable of producing high levels of 1,2 propanediol (1,2-PD) when grown on glycerol as the sole carbon source, wherein said host cell, particularly said microorganism or strain, is engineered to co-express at least one enzyme protein selected from the group consisting of glycerol dehydrogenase (gldA), dihydroxyacetone kinase (dhaK) and methylglyoxal synthase (mgsA) along with the propanediol oxidoreductase (fucO) activity, particularly by co-introducing in said host cell together with the gene encoding a propanediol oxidoreductase (fucO) activity at least one additional gene encoding an enzyme activity selected from the group consisting of glycerol dehydrogenase (gldA), dihydroxyacetone kinase (dhaK) and methylglyoxalsynthase (mgsA) such as to express said activities along with the propanediol oxidoreductase (fucO) activity.

In one embodiment, the invention provides a host cell, particularly a microorganism or strain, which is capable of producing high levels of 1,2 propanediol (1,2-PD) when grown on glycerol as the sole carbon source, wherein said host cell, particularly said microorganism or strain, is engineered to co-express a glycerol dehydrogenase (gldA) activity along with the propanediol oxidoreductase (fucO) activity, particularly by co-introducing in said host cell together with the gene encoding a propanediol oxidoreductase (fucO) activity, a gene encoding a glycerol dehydrogenase (gldA) activity such as to express said glycerol dehydrogenase (gldA) activity along with the propanediol oxidoreductase (fucO) activity.

In one embodiment, the invention provides a host cell, particularly a microorganism or strain, which is capable of producing high levels of 1,2 propanediol (1,2-PD) when grown on glycerol as the sole carbon source, wherein said host cell, particularly said microorganism or strain, is engineered to co-express a dihydroxyacetone kinase (dhaK) activity along with the propanediol oxidoreductase (fucO) activity, particularly by co-introducing in said host cell together with the gene encoding a propanediol oxidoreductase (fucO) activity, a gene encoding a dihydroxyacetone kinase (dhaK) activity such as to express said dihydroxyacetone kinase (dhaK) activity along with the propanediol oxidoreductase (fucO) activity.

In one embodiment, the invention provides a host cell, particularly a microorganism or strain, which is capable of producing high levels of 1,2 propanediol (1,2-PD) when grown on glycerol as the sole carbon source, wherein said host cell, particularly said microorganism or strain, is engineered to co-express a methylglyoxalsynthase (mgsA) activity along with the propanediol oxidoreductase (fucO) activity, particularly by co-introducing in said host cell together with the gene encoding a propanediol oxidoreductase (fucO) activity, a gene encoding a methylglyoxalsynthase (mgsA) activity such as to express said methylglyoxalsynthase (mgsA) activity along with the propanediol oxidoreductase (fucO) activity.

In one embodiment, the invention provides a host cell, particularly a microorganism or strain, which is capable of producing high levels of 1,2 propanediol (1,2-PD) when grown on glycerol as the sole carbon source, wherein said host cell, particularly said microorganism or strain, is engineered to co-express a glycerol dehydrogenase (gldA) and a dihydroxyacetone kinase (dhaK) activity along with the propanediol oxidoreductase (fucO) activity, particularly by co-introducing in said host cell together with the gene encoding a propanediol oxidoreductase (fucO) activity, genes encoding a glycerol dehydrogenase (gldA) and a dihydroxyacetone kinase (dhaK) activity such as to express said glycerol dehydrogenase (gldA) and dihydroxyacetone kinase (dhaK) activities along with the propanediol oxidoreductase (fucO) activity.

In one embodiment, the invention provides a host cell, particularly a microorganism or strain, which is capable of producing high levels of 1,2 propanediol (1,2-PD) when grown on glycerol as the sole carbon source, wherein said host cell, particularly said microorganism or strain, is engineered to co-express a glycerol dehydrogenase (gldA) and a methylglyoxalsynthase (mgsA) activity along with the propanediol oxidoreductase (fucO) activity, particularly by co-introducing in said host cell together with the gene encoding a propanediol oxidoreductase (fucO) activity, genes encoding a glycerol dehydrogenase (gldA) and a methylglyoxalsynthase (mgsA) activity such as to express said glycerol dehydrogenase (gldA) and methylglyoxalsynthase (mgsA) activities along with the propanediol oxidoreductase (fucO) activity.

In one embodiment, the invention provides a host cell, particularly a microorganism or strain, which is capable of producing high levels of 1,2 propanediol (1,2-PD) when grown on glycerol as the sole carbon source, wherein said host cell, particularly said microorganism or strain, is engineered to co-express a dihydroxyacetone kinase (dhaK) and a methylglyoxalsynthase (mgsA) activity along with the propanediol oxidoreductase (fucO) activity, particularly by co-introducing in said host cell together with the gene encoding a propanediol oxidoreductase (fucO) activity, genes encoding a dihydroxyacetone kinase (dhaK) and a methylglyoxalsynthase (mgsA) activity such as to express said dihydroxyacetone kinase (dhaK) and methylglyoxalsynthase (mgsA) activities along with the propanediol oxidoreductase (fucO) activity.

In one embodiment, the invention provides a host cell, particularly a microorganism or strain, which is capable of producing high levels of 1,2 propanediol (1,2-PD) when grown on glycerol as the sole carbon source, wherein said host cell, particularly said microorganism or strain, is engineered to co-express a glycerol dehydrogenase (gldA), a dihydroxyacetone kinase (dhaK) and a methylglyoxalsynthase (mgsA) activity along with the propanediol oxidoreductase (fucO) activity, particularly by co-introducing in said host cell together with the gene encoding a propanediol oxidoreductase (fucO) activity, genes encoding a glycerol dehydrogenase (gldA), a dihydroxyacetone kinase (dhaK) and a methylglyoxalsynthase (mgsA) activity such as to express said glycerol dehydrogenase (gldA), dihydroxyacetone kinase (dhaK) and methylglyoxalsynthase (mgsA) activities along with the propanediol oxidoreductase (fucO) activity.

In one embodiment, the invention provides a host cell, particularly a microorganism or strain, which is capable of producing high levels of 1,2 propanediol (1,2-PD) when grown on glycerol as the sole carbon source, wherein said host cell, particularly said microorganism or strain, is engineered to co-express a glycerol dehydratase activity along with the propanediol oxidoreductase (fucO) activity, particularly by co-introducing in said host cell together with the gene encoding a propanediol oxidoreductase (fucO) activity, genes encoding a glycerol dehydratase activity such as to express said glycerol dehydratase activity along with the propanediol oxidoreductase (fucO) activity.

In one embodiment, the invention provides a host cell, particularly a microorganism or strain, which is capable of producing high levels of 1,2 propanediol (1,2-PD) when grown on glycerol as the sole carbon source, wherein said host cell, particularly said microorganism or strain, is engineered to co-express an aldo-keto-reductase activity along with the propanediol oxidoreductase (fucO) activity, particularly an aldo-keto-reductase activity, which is contributed by a gene, particularly a microbial gene, selected from the group consisting of dkgA, dkgB, yeaE and yghZ, particularly by co-introducing in said host cell together with the gene encoding a propanediol oxidoreductase (fucO) activity, genes encoding an aldo-keto-reductase activity, particularly a microbial gene, selected from the group consisting of dkgA, dkgB, yeaE and yghZ such as to express said aldo-keto-reductase activity along with the propanediol oxidoreductase (fucO) activity.

In one embodiment, the invention provides a host cell, particularly a microorganism or strain, which is capable of producing high levels of 1,2 propanediol (1,2-PD) when grown on glycerol as the sole carbon source, wherein said host cell, particularly said microorganism or strain, has been engineered through recombinant DNA techniques.

In one embodiment, the invention provides a host cell, particularly a microorganism or strain according to the invention and as described herein before, which is capable of producing high levels of 1,2 propanediol (1,2-PD) when grown on glycerol as the sole carbon source, wherein said host cell, particularly a microorganism or strain, is defective in arabinose metabolism. In on embodiment, said host cell, particularly said microorganism or strain, is defective in arabinose metabolism due to a reduced or missing ribulose kinase activity.

In one embodiment, the invention provides a host cell, particularly a microorganism or strain according to the invention and as described herein before, which is capable of producing high levels of 1,2 propanediol (1,2-PD) when grown on glycerol as the sole carbon source, wherein said host cell, particularly said microorganism or strain, is defective in the metabolism of methylglyoxal. In one embodiment, said host cell, particularly said microorganism or strain, is defective in the metabolism of methylglyoxal due to a reduced or missing enzyme activity selected from the group consisting of glyoxylase system I, glyoxylase system II, lactate dehydrogenase A, glyoxylase system III, aldehyde dehydrogenase A activity, but especially due to a reduced or missing glyoxylase system I activity.

In one embodiment, the invention provides a host cell, particularly a microorganism or strain according to the invention and as described herein before, which is capable of producing high levels of 1,2 propanediol (1,2-PD) when grown on glycerol as the sole carbon source, wherein said host cell, particularly said microorganism or strain, is defective in the metabolism of dihydroxyacetonphosphate. In one embodiment, said host cell, particularly said microorganism or strain, is defective in the metabolism of dihydroxyacetonphosphate due to a reduced triosephosphate isomerase activity.

In one embodiment, the invention provides a host cell, particularly a microorganism or strain according to the invention and as described herein before, wherein said microorganism is E. coli.

In one embodiment, the invention provides a method for the preparation of 1,2-propanediol whereby a host cell, particularly a microorganism strain according to the invention is grown in an appropriate growth medium containing a simple carbon source, particularly a crude glycerol preparation, after which the 1,2-propanediol produced are recovered and, if necessary, purified.

In particular, the invention provides a method of producing 1,2-propanediol by growing a host cell, particularly a microorganism or strain according to the invention, on a non-fermentable carbon substrate, comprising:
i) culturing said host cell, particularly said microorganism or strain, according to the invention and as described herein before, which host cell overexpresses propanediol oxidoreductase (fucO) activity, in a medium containing a non-fermentable carbon substrate, whereby the carbon substrate is sustaining production of biomass and serves as a substrate for production of 1,2 propanediol (1,2-PD) at the same time, and the non-fermentable carbon source is metabolized by the host cell, particularly the microorganism or strain, according to the invention into 1,2-propanediol
ii) recovering the 1,2-propanediol produced according to step i); and, optionally,
iii) purifying the recovered 1,2-propanediol.

In one embodiment of the invention, said non-fermentable carbon substrate is a crude glycerol preparation, particularly a preparation containing glycerol with a purity of at least 70%, particularly of at least 75%, particularly of at least 80%, particularly of at least 85%, particularly of at least 90%, particularly of at least 95%, particularly of at least 99% and up to 100%.

In a specific embodiment, the glycerol has a degree of purity of between 80% and 90%, particularly of about 85%.

In one embodiment, the non-fermentable carbon substrate, particularly the crude glycerol preparation as described herein before, is selectively metabolized to 1,2-propanediol.

In one embodiment, the invention provides a method of producing 1,2-propanediol as described herein before, wherein a host cell, particularly a microorganism or strain, according to the invention and as described herein before is used in said process, which is engineered to overexpress propanediol oxidoreductase (fucO).

In one embodiment, the invention provides a method of producing 1,2-propanediol as described herein before, wherein a host cell, particularly a microorganism or strain, according to the invention and as described herein before is used in said process which is engineered to co-express at least one additional enzyme protein selected from the group consisting of glycerol dehydrogenase (gldA), dihydroxyacetone kinase (dhaK) and methylglyoxalsynthase (mgsA) along with the propanediol oxidoreductase (fucO) activity.

In one embodiment of the invention, a host cell, particularly a microorganism or strain, according to the invention and as described herein before is used, wherein at least one enzyme activity involved in a non-productive pathway competing with 1,2-PD production has been deactivated.

In particular, microbial mutants, particularly mutants of E. coli, are used wherein one or more of the genes encoding glyoxylase systems I and II (gloA and gloB), lactate dehydrogenase A (ldhA), glyoxylase system III (indirectly by inactivation of the master regulator rpoS), and aldehyde dehydrogenase have been deactivated.

In another embodiment, a microbial mutant or strain, particularly an E. coli mutant, is used wherein the gene encoding a gloA activity has been partially or fully inactivated:

In another embodiment, a microbial mutant or strain inactivated in arabinose metabolism is used within the process according to the invention.

In one embodiment of the invention, an E. coli strain is used as the host organism, particularly an E. coli strain MG1655 and DHSalpha, respectively.

In one embodiment of the invention, at least one of the genes encoding an enzyme activity selected from the group consisting of glycerol dehydrogenase (gldA), dihydroxyacetone kinase (dhaK) and methylglyoxalsynthase (mgsA) and propanediol oxidoreductase (fucO) is under the control of an inducible promoter, particularly an arabinose inducible promoter, particularly a paraBAD promoter.

In one embodiment of the invention, a synthetic operon is provided and used in the method according to the invention to provide a host cell, particularly a microorganism or strain, co-expressing at least one enzyme activity selected from the group consisting of glycerol dehydrogenase (gldA), dihydroxyacetone kinase (dhaK) and methylglyoxalsynthase (mgsA) activity along with the propanediol oxidoreductase (fucO) activity. In one embodiment of the invention, the genes encoding the above activities are under control of an inducible promoter, particularly an arabinose-inducible promoter, but especially a paraBAD promoter.

In one embodiment, a synthetic operon is provided comprising the gene encoding propanediol oxidoreductase (fucO) and at least one additional gene encoding an enzyme protein selected from the group consisting of glycerol dehydrogenase, dihydroxyacetone kinase and methylglyoxalsynthase (mgsA), particularly a synthetic operon comprising the genes encoding propanediol oxidoreductase (fucO), glycerol dehydrogenase, dihydroxyacetone kinase and methylglyoxalsynthase (mgsA).

In one embodiment, the synthetic operon according to the invention is under the control of an inducible promoter, particularly an arabinose-inducible promoter.

In one embodiment of the invention, the genes encoding the succession of genes transcribed upon induction from said operon is as follows: mgsA, gldA, dhaK, fucO.

The invention further relates to polynucleotide molecules or constructs, particularly plasmids and vector molecules, comprising the synthetic operon according to the invention and as described herein before and to host cells, particularly microbial host cells comprising said polynucleotide molecules.

### BRIEF DESCRIPTION OF THE DRAWINGS AND SEQUENCE LISTING

FIG. 1 illustrates inhibition of growth of wild type (black bars) and mutant strains (□gloA-mutant, grey-bars; □gloB-mutant, hatched bars) of E. coli by different amounts of methylglyoxal added to the culture broth

FIG. 2 is a schematic drawing of pathways generating 1,2-PD when sugars or glycerol are carbon substrates

FIGS. 3 & 4 illustrate maps of plasmids described within the invention

### DEFINITIONS

The term “polynucleotide” is understood herein to refer to polymeric molecule of high molecular weight which can be single-stranded or double-stranded, composed of monomers (nucleotides) containing a sugar, phosphate and a base which is either a purine or pyrimidine. The term “polynucleotide” thus primarily refers to a polymer of DNA or RNA which can be single- or double-stranded, optionally containing synthetic, non-natural or altered nucleotide bases capable of incorporation into DNA or RNA polymers. Unless otherwise indicated, a particular nucleic acid sequence of this invention also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer, et al. (1991); Ohtsuka, et al., (1985); and Rossolini, et al. (1994)). The term polynucleotide is used interchangeably with nucleic acid, nucleotide sequence and may include genes, cDNAs, and mRNAs encoded by a gene, etc.

The term “construct” refers to a plasmid, virus, autonomously replicating sequence, phage or nucleotide sequence, linear or circular, of a single- or double-stranded DNA or RNA, derived from any source, in which a number of nucleotide sequences have been joined or recombined into a unique construction which is capable of introducing a promoter fragment and DNA sequence for a selected gene product encoding an enzyme activity according to the invention along with appropriate 3′ untranslated sequence into a cell.

The term “transformation” or “transfection” refers to the acquisition of new genes in a cell after the incorporation of nucleic acid.

The term “expression” refers to the transcription and translation to gene product from a gene coding for the sequence of the gene product. In the expression, a DNA chain coding for the sequence of gene product is first transcribed to a complimentary RNA which is often a messenger RNA and, then, the thus transcribed messenger RNA is translated into the above-mentioned gene product if the gene product is a protein.

The term “plasmid” or “vector” or “cosmid” as used herein refers to an extra chromosomal element often carrying genes which are not part of the central metabolism of the cell, and usually in the form of circular double-stranded DNA molecules.

The term “regulator” used in the present specification refers to a base sequence having a functional promoter and any related transcriptional element (e.g., enhancer, CCAAT box, TATA box, SPI moiety and the like).

The term “operably linked” used in the present specification means that various regulatory elements such as a promoter, an enhancer and the like, that control the gene expression, and a gene of interest are connected in an operable state in a host cell such as to enable expression of said gene of interest. It is a well known matter to those of ordinary skill in the art that the type and kind of regulator can vary depending on the host.

The term ‘deletion’ denotes the suppression of the activity of a gene, which in general consists of a suppression of activity that can be an inactivation, an inhibition, or it can be the deletion of at least a part of the gene concerned (for example deletion of all or a part of the promoter region necessary for its expression) so that it is not expressed or non-functional or so that the expression product loses its function (for example deletion in a coding part of the gene concerned). Preferentially, the deletion of a gene is essentially the suppression of that gene, which gene can be replaced by a selection marker gene that facilitates the identification, isolation and purification of the strains according to the invention. For example, a gene may be inactivated by homologous recombination mediated by the recA-protein of e.g. E. coli (Cunningham, et al. (1980)).

Briefly, an inactivation protocol can be as follows: a linear fragment of DNA is introduced into the cell. This fragment is obtained in vitro, and comprises two regions flanking the gene, and a gene encoding a selectable gene product (generally an antibiotic-resistance gene) located between the two flanking regions. This fragment thus presents an inactivated gene. The cells that have undergone a recombination event and integrated the synthetic fragment are selected by plating on a selective growth medium. Cells that have undergone a double recombination event, in which the native gene has been replaced by the inactivated gene, are selected.

The term “carbon substrate” means any carbon source capable of being metabolized by a microorganism wherein the substrate contains at least one carbon atom.

The term “non-fermentable carbon substrate” as used in the present invention refers to carbon substrates that do not sustain redox-processes of a given organism to generate biomass in absence of exogenous electron acceptors.

1,2-propanediol (propylene glycol; 1,2-PD) is a major bulk chemical that is widely used as a component of unsaturated polyester resins, pharmaceutical formulations and cosmetics, liquid detergents, coolants and anti-freeze or de-icing fluids. Since 1,2 propanediol (1,2-PD) is optically active, enantiomerically pure preparations of 1,2 propanediol (1,2-PD) might be of special interest for medical, agricultural or physiological applications.

1,2 propanediol (1,2-PD) can be produced by microorganisms from sugars as substrates and sole carbon source. In recent years, alternative substrates such as glycerol have attracted considerable attention for use as fermentation substrate instead of e.g. sugar carbon sources. The interest in glycerol essentially is the result of significantly increased biodiesel or bio-ethanol production. Both processes generate glycerol as major by-product that makes up e.g. 10% (w/w) of the biodiesel produced.

The present invention now provides an improved biotechnological process for the production of 1,2 propanediol (1,2-PD) from a non-fermentable, inexpensive carbon substrate, particularly a crude glycerol preparation, whereby the carbon substrate is sustaining production of biomass and serves as a substrate for production of 1,2 propanediol (1,2-PD) at the same time. The present invention further provides improved microbial strains which are specifically adapted to the specific requirements of this procedure and are therefore specifically suited for use in the process according to the invention.

In a preferred embodiment, the present invention provides for bioconverting glycerol or crude glycerol preparations as a non-fermentable carbon source directly to 1,2-propanediol using a host cell, particularly a microorganism or strain, that has been engineered to contain one or more genes that are involved in the production pathway of 1,2 propanediol (1,2-PD) from glycerol. In particular, the host cell, particularly the microorganism or strain, according to the invention has been engineered by recombinant DNA techniques to produce a recombinant host cell, particularly a recombinant microorganism or strain, comprising genes involved in the metabolism of dihydroxyaceton phosphate and methylglyoxal, two key precursor compounds in the production pathway to 1,2 propanediol (1,2-PD). In particular, a host cell, particularly a microorganism or strain, is provided harbouring genes selected from the group consisting of genes encoding enzymes exhibiting a glycerol-dehydrogenase activity, a dihydroxyaceton-kinase activity, a methylgyoxal-synthase activity and a propanediol-oxidoreductase activity, which enzymes are able to convert glycerol to 1,2 propanediol (1,2-PD) with high selectivity.

In a preferred embodiment, an engineered E. coli strain, particularly a recombinant E. coli strain is used within the scope of the present invention.

It was surprisingly found within the present invention that common crude-glycerol (85% purity) from biodiesel production can be used as substrate for growth of a broad variety of organisms of different origin under oxic and anoxic conditions It could be demonstrated that most of the organisms tested were not impaired by crude glycerol compared to pure glycerol with regard to biomass production, indicating that crude glycerol can be utilised and is in general not toxic to microorganisms. Biomass production was not affected by the impurities found in crude-glycerol preparations. Crude glycerol from biodiesel-production or alternative sources can, therefore, be equally well utilised as carbon-source by microorganisms and thus can be used without further processing as a general renewable carbon source in fermentation processes for biomass production.

Crude glycerol preparations, particularly crude glycerol preparations from biodiesel or bioethanol production may therefore be used in the process according to the present invention for producing 1,2 propanediol (1,2-PD).

A first key precursor compound in the production pathway to 1,2 propanediol (1,2-PD) is dihydroxyaceton phosphate (DHAP). DHAP is converted to methylglyoxal, a 2^{nd} essential precursor compound, through the activity of a methylglyoxal synthase (mgsA). The methylglyoxal becomes finally converted into S-lactaldehyde. A so far unidentified glycerol-dehydratase activity may convert glycerol into R- or S-lactaldehyde, which is further metabolised to R- or S-1,2-PD, respectively. Whereas an endogenous reductive activity of the host cell is proposed to produce R-1,2-PD from the R-lactaldehyde, the S-lactaldehyde appears to be the substrate for the propanediol oxidoreductase (fucO), which may convert S-lactaldehyde into S-1,2-PD (Altaras, N. E., 1999; Applield and Environmental Microbiology (65), 1180-1185).

It was, therefore hypothesized that by introducing propanediol oxidoreductase (fucO) into a host organism the flexibility of the 1,2 propanediol (1,2-PD) producing network may be expanded by accepting the S-entantiomer of lactaldehyde for conversion to 1,2 propanediol (1,2-PD). Furthermore, it was concluded that propanediol oxidoreductase (fucO) activity might be necessary for production of 1,2 propanediol (1,2-PD) from glycerol independent of the methylglyoxal pathway

Accordingly, a wild-type strain that does not produce detectable amounts of 1,2 propanediol (1,2-PD) from glycerol, irrespective of the conditions for cultivation, was supplemented with a polynucleotide comprising a nucleotide sequence encoding a propanediol oxidoreductase (fucO) activity.

In one embodiment of the invention, the gene encoding a propanediol oxidoreductase (fucO) activity was cloned into a host organism which does not produce detectable amounts of 1,2 propanediol (1,2-PD) from glycerol and over-expressed in said host in minimal medium containing glycerol under oxic and semi anoxic conditions. Overexpression of propanediol oxidoreductase (fucO) activity resulted in production of 1,2 propanediol (1,2-PD).

A further key precursor compound in the production pathway to 1,2 propanediol (1,2-PD) is dihydroxyacetone phosphate (DHAP). In one embodiment of the invention, an alternative pathway to yield dihydroxyacetone phosphate as precursor for 1,2 propanediol (1,2-PD)-synthesis is engineered into a microbial strain, particularly into an E. coli strain, which pathway produces the essential precursor DHAP independent of the endogenous regulatory network acting on glycerolphosphate kinase (glpK).

In particular, a DNA molecule comprising a nucleotide sequence encoding a glycerol dehydrogenase (gldA) and dihydroxyacetone kinase (dhaK) activity is introduced in a host organism, particularly a E. coli host. The gene encoding the glycerol dehydrogenase (gldA) may be isolated from an E. coli strain, particularly an E. coli K12, and cloned into a suitable plasmid.

In one embodiment, the gene encoding the glycerol dehydrogenase (gldA) may be cloned into a suitable plasmid along with a gene encoding dihydroxyacetone kinase (dhaK) activity. The gene encoding the dihydroxyacetone kinase (dhaK) activity may be isolated from a Citrobacter strain, particularly a Citrobacter freundii strain.

In another embodiment of the invention, the glycerol dehydrogenase (gldA) gene is cloned into a suitable plasmid independent of and separate from the glycerol dehydrogenase (gldA).

In one embodiment of the invention, the introduced coding sequences encoding a glycerol dehydrogenase (gldA) and/or a dihydroxyacetone kinase (dhaK) activity are under control of an inducible promoter, particularly an arabinose inducible promoter (paraBAD).

The genes of this alternative pathway to yield dihydroxyacetone phosphate encoding the glycerol dehydrogenase (gldA) and the dihydroxyacetone kinase (dhaK) activity, respectively, may be introduced into a wild-type host organism together with a polynucleotide comprising the nucleotide sequence encoding the propanediol oxidoreductase (fucO) activity, either separately as individual expression cassettes, wherein the coding sequence is under control of its own promoter and termination signal, which expression cassettes may either be located on different plasmids or on a single plasmid, or in form of a synthetic operon comprising two or more of said genes under the control of common promoter and termination sequences.

In one embodiment of the invention, the genes encoding the glycerol dehydrogenase (gldA) and dihydroxyacetone kinase (dhaK) activity are cloned to a single plasmid which already comprises a gene encoding the propanediol oxidoreductase (fucO) activity to create a plasmid comprising a gene encoding a glycerol dehydrogenase (gldA) along with the propanediol oxidoreductase (fucO) activity, or a plasmid comprising a gene encoding a dihydroxyacetone kinase (dhaK) along with the propanediol oxidoreductase (fucO) activity.

In one embodiment, a plasmid is created, which comprises a gene encoding a glycerol dehydrogenase (gldA) and a dihydroxyacetone kinase (dhaK) along with the propanediol oxidoreductase (fucO) activity.

The various gene sequences encoding the different enzyme activities may be arranged on the plasmid such as to create a synthetic operon, wherein two or more genes are arranged under the control of common regulatory sequences including promoter and polyadenylation sequences. In one embodiment, the synthetic operon is under control of an inducible promoter, particularly an arabinose-inducible promoter.

DHAP is the initial intermediate in the pathway generating 1,2 propanediol (1,2-PD). Triosephosphateisomerase (tpi) of the glycolytic pathway competes with methylglyoxal synthase for DHAP. In order to drive 1,2 propanediol (1,2-PD) production, mgsA encoding methylglyoxal synthase may be incorporated in the synthetic operon in order to shift the balance towards 1,2 propanediol (1,2-PD) production.

In one embodiment of the invention, an extended synthetic operon is therefore provided comprising in addition to the genes involved in the dihydroxyaceton phosphate pathway an additional gene involved the production of methylglyoxal, particularly a methylgyoxal-synthase gene, particularly a methylgyoxal-synthase gene of E. coli.

The resulting plasmid(s) is(are) then introduced in a host cell, particularly a microbial host cell or strain, which is unable of producing detectable amounts of 1,2 propanediol (1,2-PD) from glycerol, irrespective of the conditions for cultivation, particularly in an E. coli strain.

In one embodiment of the invention, the host organism has no active arabinose metabolism or has previously been inactivated in arabinose metabolism by deleting or inactivating at least one of the essential genes involved in the arabinose metabolism such as, for example, the gene encoding ribulose-kinase activity (araB). The corresponding strains are cultivated in minimal medium containing glycerol under oxic and semi anoxic conditions, and the 1,2 propanediol (1,2-PD) is isolated from the supernatants and analysed.

Overexpression of the propanediol oxidoreductase gene (fucO) results in production of 1,2 propanediol (1,2-PD) from crude glycerol preparations. The amounts of 1,2 propanediol (1,2-PD) can be increased by co-expression of a dihydroxyacetone kinase (dhaK) and/or a glycerol dehydrogenase gene (gldA) together with the propanediol oxidoreductase gene (fucO). Further improvements may be achieved by co-expression of a dihydroxyacetone kinase (dhaK) and/or a glycerol dehydrogenase gene (gldA) and/or a methylgyoxal-synthase gene (mgsA) together with the propanediol oxidoreductase gene (fucO) and/or by the use of host cells, particularly microbial host cells or strains, which are defective in at least one of the non-productive pathways competing for key precursor compounds in the 1,2-propanediol production pathway.

The arrangement of the genes involved in catalysis in the described manner as 5′-mgsA, gldA, dahK, fucO-3′ is preferred, however the invention is not restricted to this specified arrangement. Any order of the described genes might be suitable for 1,2 propanediol (1,2-PD) production.

The pathway was demonstrated to be specific for the production of the 1,2 propanediol (1,2-PD) isomer of propanediol, since no 1,3-propanediol was detected.

Methods of obtaining desired genes from a bacterial genome are common and well known in the art of molecular biology. For example, if the sequence of the gene is known, suitable genomic libraries may be created by restriction endonuclease digestion and may be screened with probes complementary to the desired gene sequence. Once the sequence is isolated, the DNA may be amplified using standard primer directed amplification methods such as polymerase chain reaction (PCR) (U.S. Pat. No. 4,683,202) to obtain amounts of DNA suitable for transformation using appropriate vectors. Alternatively, cosmid libraries may be created where large segments of genomic DNA (35-45 kb) may be packaged into vectors and used to transform appropriate hosts. Cosmid vectors are unique in being able to accommodate large quantities of DNA. Generally, cosmid vectors have at least one copy of the cos DNA sequence which is needed for packaging and subsequent circularization of the foreign DNA. In addition to the cos sequence these vectors will also contain an origin of replication such as ColE1 and drug resistance markers such as a gene resistant to ampicillin or neomycin. Methods of using cosmid vectors for the transformation of suitable bacterial hosts are well described in Sambrook et al., (1989). Typically to clone cosmids, foreign DNA is isolated and ligated, using the appropriate restriction endonucleases, adjacent to the cos region of the cosmid vector. Cosmid vectors containing the linearized foreign DNA are then reacted with a DNA packaging vehicle such as bacteriophage 1. During the packaging process the cos sites are cleaved and the foreign DNA is packaged into the head portion of the bacterial viral particle. These particles may then be used to transfect suitable host cells such as E. coli. Once injected into the cell, the foreign DNA circularizes under the influence of the cos sticky ends. In this manner large segments of foreign DNA can be introduced and expressed in recombinant host cells.

Once a gene has been isolated and its sequences put into the public domain, the references given, for example, on GenBank for these known genes can be used by those skilled in the art to determine the equivalent genes in other organisms, bacterial strains, yeasts, fungi, mammals and plants, etc. This routine work is advantageously performed using consensus sequences that can be determined using sequence alignments with genes from other micro-organisms, and by designing degenerate probes by means of which the corresponding gene can be cloned in another organism. These routine techniques of molecular biology are well known to the art and are described, for example, in Sambrook et al. (1989).

In another embodiment the present invention provides a variety of vectors and transformation and expression cassettes suitable for the cloning, transformation and expression of the enzymatic activities according to the invention.

Said vector may be, for example, a phage, plasmid, viral or retroviral vector. Retroviral vectors may be replication competent or replication defective. In the latter case, viral propagation generally will occur only in complementing host/cells.

The polynucleotides or genes of the invention may be joined to a vector containing selectable markers for propagation in a host. Generally, a plasmid vector is introduced in a precipitate such as a calcium phosphate precipitate or rubidium chloride precipitate, or in a complex with a charged lipid or in carbon-based clusters, such as fullerens. Should the vector be a virus, it may be packaged in vitro using an appropriate packaging cell line prior to application to host cells.

In a more preferred embodiment of the vector of the invention the polynucleotide is operatively linked to expression control sequences allowing expression in prokaryotic or eukaryotic cells or isolated fractions thereof.

Expression of said polynucleotide comprises transcription of the polynucleotide, preferably into a translatable mRNA. Regulatory elements ensuring expression in eukaryotic cells such as a bacterial or fungal cells, an insect cells, an animal cells, mammalian cells or a human cells, but particularly bacterial or fungal cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast. Beside elements which are responsible for the initiation of transcription such regulatory elements may also comprise transcription termination signals, downstream of the polynucleotide.

In this context, suitable expression vectors are known in the art such as Okayama-Berg cDNA expression vector pcDVl (Pharmacia), pCDM8, pRc/CMV, pcDNA1, pcDNA3 (In-vitrogene), pSPORT1 (GIBCO BRL), pSE380 (In-vitrogene), or any pBR322 or pUC18-derived plasmids. Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, (1989) and Ausubel (1994). Alternatively, the polynucleotides and vectors of the invention can be reconstituted into liposomes for delivery to target cells.

The present invention furthermore relates to a host cell genetically engineered with the polynucleotide of the invention, the gene of the invention or the vector of the invention. Suitable host cells for the recombinant production of 1,2-propanediol may be either prokaryotic or eukaryotic and will be limited only by the host cell ability to express active enzymes.

Said host cell may be a prokaryotic or eukaryotic cell. The polynucleotide or vector of the invention which is present in the host cell may either be integrated into the genome of the host cell or it may be maintained extrachromosomally. In this respect, it is also to be understood that the present invention also relates to recombinant DNA molecules that can be used for “gene targeting” and/or “gene replacement”, for restoring a mutant gene or for creating a mutant gene via homologous recombination; see for example Mouellic, (1990); Joyner, Gene Targeting, A Practical Approach, Oxford University Press.

The host cell can be any prokaryotic or eukaryotic cell, such as a bacterial or fungal cell, an insect cell, an animal cell, a mammalian cell or a human cell, but particularly a bacterial or fungal cell. Preferred hosts will be those typically useful for production of glycerol or 1,2-propanediol. Preferred fungal cells are, for example, those of the genus Aspergillus, Saccharomyces, Schizosaccharomyces, Zygosaccharomyces, Pichia, Kluyveromyces, Candida, Hansenula, Debaryomyces, Mucor and Torulopsis, in particular those of the species S. cerevisiae. The term “prokaryotic” is meant to include all bacteria and archaea which can be transformed or transfected with a polynucleotide for the expression of an enzyme activity according to the present invention. Prokaryotic hosts may include gram negative as well as gram positive bacteria such as, for example Citrobacter, Enterobacter, Clostridium, Klebsiella, Aerobacter, Lactobacillus, Methylobacter, Escherichia, Salmonella, Bacillus, Streptomyces and Pseudomonas. Most preferred in the present invention are E. coli, S. typhimurium, Serratia marcescens and Bacillus subtilis, Klebsiella species and Saccharomyces species, but particularly E. coli species.

Specific examples thereof include Escherichia coli MG1655 (ATCC 700926; Bachmann, B., pp. 2460-2488 in Neidhardt et a1.1996), Escherichia coli XL1-Blue MRF′ [manufactured by Stratagene, Strategies, 5, 81 (1992)], Escherichia coli C600 [Genetics, 39, 440 (1954)], Escherichia coli Y1088 [Science, 222, 778 (1983)], Escherichia coli Y1090 [Science, 222, 778 (1983)], Escherichia coli NM522 [J. Mol. Biol., 166, 1 (1983)], Escherichia coli K802 [J. Mol. Biol., 16, 118 (1966)], Escherichia coli JM109 [Gene, 38, 275 (1985)], Escherichia coli DH5α [J. Mol. Biol., 166, 557 (1983)], and the like.

Further improvements may be achieved in 1,2-PD production by the use of suitable microbial mutants, wherein some or all enzyme activities involved in non productive pathways have been reduced or eliminated. For example, the enzymatic activities encoded by mgsA and tpi compete for DHAP. Methylglyoxal-synthase activity (MgsA) was shown to be inactivated by diphosphate (Hopper, D. J. 1972). In order to improve conversion of DHAP into methylglyoxal, a phosphate-insensitive mutant of MgsA can be identified by screening variant libraries obtained by any method generating variation within coding-sequences of mgsA, e.g. error-prone PCR. Microbial strains, particularly E. coli clones, previously inactivated in triosephosphate isomerase (tpiA) and endogenous mgsA, are transformed with plasmid libraries of mgsA-variants and grown on non-selective solid-media. By replica-plating of the initial transformants (plate A) on agar-plates containing high concentrations of glycerol or DHAP (plate B) or high-concentrations of diphosphate and glycerol or DHAP (plate C), clones that can grow on plate A and B, but not on plate C will be selected. These clones encode mgsA-variants with significant activity that produce toxic levels of methylglyoxal in presence of high concentrations of phosphate.

Triosephosphate isomerase mutants may be generated as described for methylglyoxal-synthase mutants. Tpi-mutants that are significantly impaired concerning growth kinetics are identified comparing growth kinetics on complex medium (e.g. Luria Broth, LB), glucose and glycerol. The mutant of interest shows slower or no growth compared to the unmodified strain with glycerol as sole source for carbon and energy, whereas growth kinetics with LB or glucose as carbon-source is unaffected.

Two other major routes for the detoxification of MG exist that are productive in terms of 1,2 PD biosynthesis. They are catalysed by so called MG-reductases as initial step. Several enzymes are proposed to encode this activity that should be strengthened by the inactivation of the competing, non-productive pathways.

In one embodiment of the invention, microbial mutants, particularly mutants of E. coli, are constructed wherein one or more of the genes encoding glyoxylase systems I and II (gloA and gloB), lactate dehydrogenase A (ldhA), glyoxylase system III (indirectly by inactivation of the master regulator rpoS), and aldehyde dehydrogenase A (aldA) have been inactivated such as to significantly reduce or completely inhibit expression of functional enzyme activities, through, for example, single gene knock-outs. This way, a microbial mutant can be obtained which has one or more of the mentioned genes inactivated, particularly a mutant wherein 2, particularly 3, particularly 4, particularly 5 of the genes selected from the group consisting of the genes encoding glyoxylase system I (gloA), glyoxylase systems II (gloB), lactate dehydrogenase A (ldhA), glyoxylase system III (indirectly by inactivation of the master regulator rpoS), and aldehyde dehydrogenase A (aldA) are inactivated.

In one embodiment of the invention, a microbial mutant, particularly an E. coli mutant, is provided wherein the gene encoding a gloA activity has been partially or fully inactivated:

In one embodiment of the present invention, a host cell, particularly a microorganism or strain, particularly a prokaryotic microorganism, e.g. E. coli, is inactivated in its ability to metabolise methylglyoxal (MG) into D- and/or L-lactate (MG-to-lactate metabolism). This is achieved by e.g. inactivation of glyoxylase A, preferably in combination with inactivation of one or more of the genes encoding glyoxylase B, the alternative sigma-factor rpoS and aldehyde-reductase A. In a preferred embodiment, a strain is deficient of all the above listed activities and/or genes.

In another embodiment, a strain inactivated in arabinose metabolism and in MG-to lactate metabolism, e.g. by inactivation of glyoxylase A, is transformed with a polynucleotide comprising the genes encoding an an enzyme activity selected from the group consisting of methylglyoxalsynthase (mgsA), glycerol dehydrogenase (gldA), dihydroxyacetone kinase (dhaK) and propanediol oxidoreductase (fucO) activity, particularly with plasmid pDP_mgdf, whereas the arrangement of the genes in the synthetic operon is not limited to that shown in plasmid pDP_mgdf, but can be in any order. The invention also refers to a strain not disabled in arabinose metabolism, e.g. wild type E. coli.

In another embodiment, a strain preferably but not necessarily inactivated in MG-to-lactate metabolism is transformed with plasmid-encoded genes that confer aldo-keto-reductase activity. The activity encoding genes are taken from a group of E. coli genes comprising dkgA, dkgB, yeaE and yghZ.

In a preferred embodiment, a strain expressing glycerol dehydrogenase (gldA), dhydroxyacetone kinase, propanediol oxidoreductase (fucO) and methylglyoxal synthase (e.g. by plasmid pDP_mgdf) is transformed with a plasmid encoding aldo-keto-reductase activity (e.g. DkgA of E. coli) and cultivated in a medium containing crude glycerol as carbon source.

In a specially preferred embodiment, a strain inactivated in MG-to-lactate metabolism and expressing glycerol dehydrogenase (gldA), dhydroxyacetone kinase, propanediol oxidoreductase (fucO) and methylglyoxal synthase (e.g. encoded on plasmid pDP_mgdf) expresses aldo-keto-reductase activity (e.g. dkgA of E. coli encoded on plasmid pCR2.1) and cultivated in a medium containing crude glycerol as carbon source.

The invention also refers to a strain not disabled in arabinose metabolism, and/or to strains expressing relevant enzyme activities cited within this invention from the chromosome of the microorganism.

A polynucleotide coding for an enzyme activity according to the present invention can be used to transform or transfect the host cell using any of the techniques commonly known to those of ordinary skill in the art.

The technique preferentially used to introduce these genes into the strain is electroporation, which is well known to those skilled in the art. Briefly, an electroporation protocol can be as follows: the heterologous genes of interest are cloned in an expression vector between a promoter and a terminator. This vector also possesses an antibiotic resistance gene to select cells that contain it and a functional replication origin in the host strain so it can be maintained. The protocol requires the preparation of electrocompetent host cells, which are then converted by electroporation by the vector. According to the invention, the genes introduced by electroporation are preferentially the genes according to the invention encoding an enzyme activity selected from the group consisting of glycerol dehydrogenase (gldA), dihydroxyacetone kinase (dhaK), methylglyoxalsynthase (mgsA) and propanediol oxidoreductase (fucO) activity.

Methods for preparing fused, operably linked genes and expressing them in bacteria or animal cells are well-known in the art (Sambrook, supra). The genetic constructs and methods described therein can be utilized for expression of polypeptides of the invention in, e.g., prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes, which are capable of providing phenotypic selection of the transformed cells. The transformed prokaryotic hosts can be grown in fermentors and cultured according to techniques known in the art to achieve optimal cell growth.

Typically, cells are grown at 30° C. in appropriate media. Preferred growth media in the present invention are defined or synthetic, e.g. minimal medium M9 containing glycerol as carbon source. Common commercially prepared media such as Luria Bertani (LB) broth, Sabouraud Dextrose (SD) broth or Yeast Malt Extract (YM) broth may also be used and the appropriate medium for growth of the particular microorganism will be known by a person skilled in the art of microbiology or fermentation science. The use of agents known to modulate catabolite repression directly or indirectly, e.g., cyclic adenosine 2′:3′-monophosphate or cyclic adenosine 2′:5′-monophosphate, may also be incorporated into the reaction media. Similarly, the use of agents known to modulate enzymatic activities (e.g., sulphites, bisulphites and alkalis) that lead to enhancement of 1,2-PD production may be used in conjunction with or as an alternative to genetic manipulations.

Suitable pH ranges for the fermentation are between pH 5.0 to pH 9.0, where pH 6.0 to pH 8.0 is preferred as range for the initial condition. Reactions may be performed under aerobic, microaerobic or anaerobic conditions where aerobic or microaerobic conditions are preferred.

Batch and Continuous Fermentations: The present process uses a batch method of fermentation. A classical batch fermentation is a closed system where the composition of the media is set at the beginning of the fermentation and not subject to artificial alterations during the fermentation. Thus, at the beginning of the fermentation the media is inoculated with the desired organism or organisms and fermentation is permitted to occur adding nothing to the system. Typically, however, a batch fermentation is “batch” with respect to the addition of the carbon source and attempts are often made at controlling factors such as pH and oxygen concentration. The metabolite and biomass compositions of the batch system change constantly up to the time the fermentation is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of end product or intermediate. A variation on the standard batch system is the Fed-Batch fermentation system which is also suitable in the present invention. In this variation of a typical batch system, the substrate is added in increments as the fermentation progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO.sub.2. Batch and Fed-Batch fermentations are common and well known in the art and examples may be found in Brock, supra. It is also contemplated that the method would be adaptable to continuous fermentation methods. Continuous fermentation is an open system where a defined fermentation media is added continuously to a bioreactor and an equal amount of conditioned media is removed simultaneously for processing. Continuous fermentation generally maintains the cultures at a constant high density where cells are primarily in log phase growth. Continuous fermentation allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, supra. The present invention may be practiced using either batch, fed-batch or continuous processes and that any known mode of fermentation would be suitable. Additionally, it is contemplated that cells may be immobilized on a substrate as whole cell catalysts and subjected to fermentation conditions for 1,2-propanediol production.

The 1,2 propanediol product can then be isolated from the grown medium or cellular lysates. The isolation and purification of the microbially or otherwise produced propanediols may be by any conventional means. Methods for the purification of propanediols from fermentation or cultivation media are known in the art. For example, propanediols can be obtained from cell media by subjecting the reaction mixture to extraction with an organic solvent, distillation and column chromatography (U.S. Pat. No. 5,356,812). A particularly good organic solvent for this process is cyclohexane (U.S. Pat. No. 5,008,473).

For industrial applications, purification of 1,2-propanediol from large volumes of fermentor broth requires non-laboratory scale methods. Difficulties to be overcome include removal of cell matter form the broth (clarification), concentration of 1,2-propanediol either by extraction or water removal and separation of residual impurities from the partially purified monomer. Broth clarification will typically proceed either by filtration, centrifugation or crossflow microfiltration. Suitable filters are manufactured for example by Millipore (Millipore Corporation, 80 Ashby Road, Bedford, Mass.) or Filmtec (Dow Chemical Co.). Centrifugation effectively removes the bulk of the cells, but, depending upon the nature of the broth, does not always achieve complete cell removal. Crossflow microfiltration yields extremely clear filtrate. The concentrate is a slurry rather than a high-solids cake. The skilled person will be able to adapt the clarification method most appropriate for the fermentation apparatus and conditions being employed. Water reduction of the clarified broth is complicated by the high solubility of 1,2-propanediol in water. Extraction of 1,2-propanediol from the clarified broth may be accomplished by a variety of methods, including evaporation/distillation, membrane technology, extraction by organic solvent and adsorption. Rotary evaporators may be used to initially reduce water volume in the clarified broth. This method has enjoyed good success in Applicants' hands. Precipitation of extraneous proteins and salts do not appear to affect 1,2-propanediol recovery Membrane technology may be used either separately or in conjunction with evaporation. Suitable membranes will either (i) allow passage of 1,2-propanediol, retaining water and other feed molecules (ii) allow passage of water and other molecules, retaining 1,2-propanediol or (iii) allow passage of water and 1,2-propanediol while retaining other molecules. In the present invention method (iii) is preferred. Particularly useful, are reverse osmosis membranes such as SW-30 2540 (Filmtec, Dow Chemical Co.) and the DL and SH series of reverse osmosis membranes made by Millipore (Millipore Corporation, Bedford, Mass.). Following evaporation and membrane concentration, partially purified 1,2-propanediol may be extracted into organic solvents. Suitable solvent will include alcohols such as tert-amyl alcohol, cyclopentanol, octanol, propanol, methanol, and ethanol. Non alcohols may also be used such as octanone, cyclohexane and valeraldehyde. Within the context of the present invention, alcohols are preferred and ethanol is most preferred. Alternatively 1,2-propanediol may be further concentrated by adsorption to various industrial adsorbents. Activated carbon and polycyclodextrin such as those produced by the American Maize Products Company are particularly suitable. Following either extraction or adsorption, partially purified 1,2-propanediol must be refined. Refining may be accomplished by electrodialysis (particularly useful for desalting) which utilizes a combination of anion and cation exchange membranes or biopolar (anion and cation) membranes (see for example, Grandison, Alistair S., (1996)) A preferred method of refining in the present invention is distillation. Distillation may be done in batch where the operating pressure is ambient or below, e.g. about 25 in. Hg of vacuum. Monitoring of distillation indicated that materials evaporated in the order of first to last beginning with light organics, water, diols including 1,2-propanediol and finally heavy materials such as glycerol and precipitated solids.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently claimed subject matter.

All manipulations and techniques necessary to construct and propagate strains described in this invention are known to those skilled in the art. Technical details are described e.g. in Ausubel et al 1995; Sambrook, J, 2001 and Miller, J. H. 1992 and in relevant publications cited within this invention.

### Example 1

### General Methodology

### 1.1 Strain Cultivation

E. coli was cultured in a defined minimal medium that was designed to contain low levels of phosphate, since phosphate is a known inhibitor of methyglyoxal synthase. Per liter, the medium contained:

### (NH₄)₂SO₄—3 g

Yeast extract—0.2 g

### CoCl₂—1.9 e-6 g

Bis-(2-hydroxyethyl)-imino-tris-(hydroxymethyl)-methane—10 g

### KH₂PO₄—0.002 g

### K₂HPO₄—0.0085 g

### MgSO₄—0.225 g

Trace element solution [Pfennig, 1966]—1 ml

If appropriate, antibiotics were added to the medium. Concentrations used were gentamycin, 5 μg/l, ampicillin, 10 μg/l.

Crude glycerol was obtained from biodiesel production and had a purity of 85%.

E. coli strains were routinely propagated in cultivation tubes (total volume 30 ml. Inoculum 5 ml) or glass bottles sealed with rubber-stoppers (total volume 12 ml, inoculum 10 ml) for creating semi-anoxic conditions. The term “cultivation under oxic conditions” implies cultivation in non-sealed containments with agitation. Semi-anoxic in that context means cultivation of strains without agitation in medium that was prepared under oxic conditions and in closed containments, e.g. bottles sealed with rubber-stoppers upon inoculation to avoid diffusing in of external oxygen. Cultivation times varied between 2 and 5 days, for oxic and semi-anoxic conditions, respectively. In general, experiments were stopped when optical density failed to increase further.

### 1.2 Analysis of 1,2-PD Formation

Levels of 1,2-PD in supernatants in culture broth were determined by three different methods, comprising a colorimetric assay, HPLC and GC-MS. Whereas HPLC using a cation-exchange column did not allow for differentiation between 1,2- and 1,3-isomers of propanediol, the colorimetric assay was specific for 1,2-PD. GC-MS analysis allowed for simultaneous quantification of both isomers separately. Detection levels were 0.5 g/l for HPLC-analysis, 50 mg/l for the colorimetric assay, and 10 mg/l for GC-MS analysis.

For routine analysis, 1,2-PD in supernatants was determined by a colorimetric method described by Jones and Riddick {Jones, 1957}. Basically, sulphuric acid is added to cell-free supernatant sample, mixed and heated. Thereafter, a ninhydrin solution and sodium-bisulfate is added, mixed and incubated for one hour. Another aliquot of sulphuric acid is added and the absorption at 595 nm, which is equivalent to the concentration of 1,2-PD, is recorded.

For quantitative analysis, 1,2-PD in samples was measured by GC-MS analysis. 1,2-PD was identified by identical retention times compared to authentic material and by mass-fingerprinting.

### Example 2

### Construction of Recombinant Organisms

2.1 Strains and Plasmids Used in this Invention

E. coli MG1655 (F-lambda-ilvG-rfb-50 rph-1) and DHSalpha (F⁻, φ80dlacZΔM15, Δ(lacZYA-argF) U169, deoR, recA1, endA1, hsdR17(rk⁻, mk⁺), phoA, supE44, λ⁻, thi-1, gyrA96, relA1) and derivatives thereof were used as host for the production of 1,2-PD. Furthermore, genomic DNA of MG1655 provided the source for amplification of relevant genes. Genomic DNA from Citrobacter freundii (DSM30040) was used as template for the amplification of the dhaK gene.

### 2.2 Isolation and Cloning of Genes

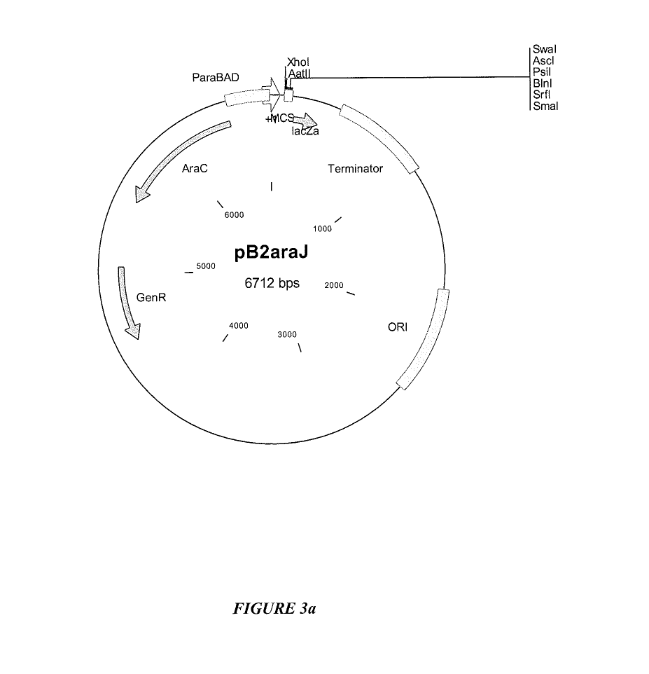

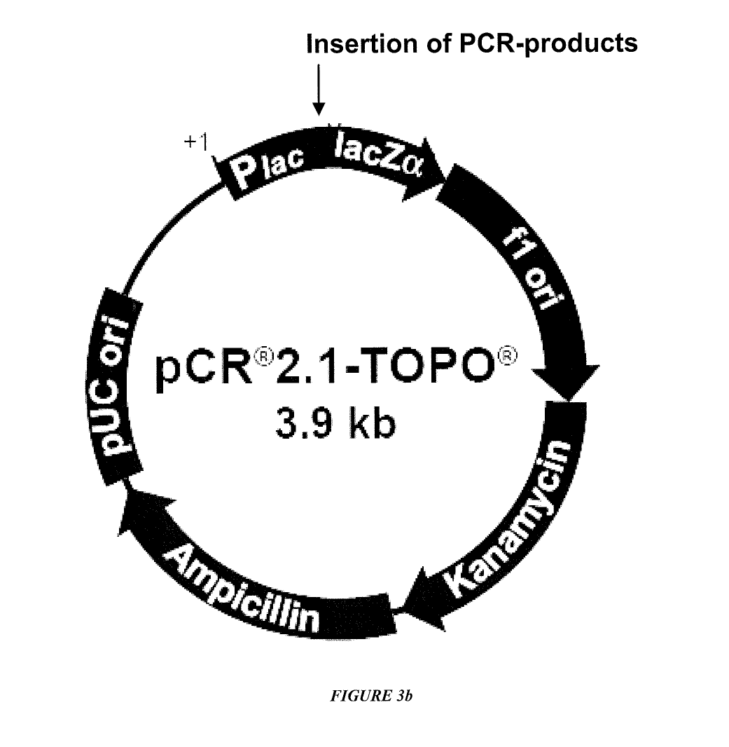

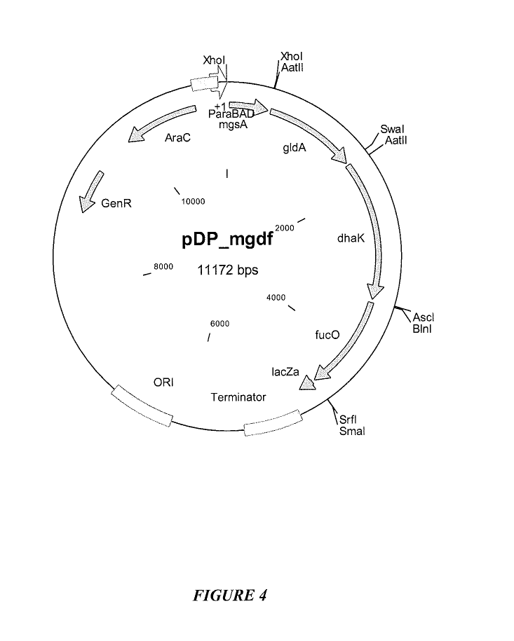

As first step, the E. coli gene for glycerol dehydrogenase gldA (SEQ ID NO: 29) was introduced in plasmid pB2araJ (FIG. 3a) or plasmid pCR2.1 (FIG. 3b). All primers used for amplification of genes of interest are listed in Table 1. Primers gldH_for1 and gldH_rev1 were used to amplify the 1,104-bp gldA Fragment from E. coli. The gel-purified PCR-fragment was inserted into the AatII-SwaI site of the pB2araJ vector, to give pDP_g. In this plasmid gldA-expression is under control of promotor paraBAD, allowing a tightly regulated, inducible expression by L-arabinose as described by Guzman et al. (Guzman, L. M., 1995). The dhaK-gene was amplified from C. freundii using the primers dhaK_for1 and dhaKrev1. The obtained 1659-nt sequence is shown in SEQ ID NO: 27, the corresponding protein sequence in SEQ ID NO: 30. The gel-purified Fragment of dhaK was inserted into the Swal-AscI site of pDP_g, resulting in pDP_gd, which cotranscribes both gldA and dhaK. The primers fucO_for1 and fucO_rev1 were used to amplify the 1152-nt gene encoding propanediol oxidoreductase (fucO) from E. coli (SEQ ID NO: 31), which was ligated into the AvrII-SmaI-site of pDP_gd, to obtain the plasmid pDP_gdf cotranscribing gldA, dhaK and fucO. The 468-bp fragment of mgsA encoding the E. coli methylglyoxalsynthase as shown in SEQ ID NO: 32 was amplified from E. coli using the primers mgsA_xhoI_for and mgsA_xhoI_rev and introduced in sense-orientation into the XhoI-site to obtain pDP_mgdf. The succession of genes transcribed upon induction from plasmid pDP_mgdf is thus as follows: mgsA, gldA, dhaK, fucO and lacZ-alpha, whereas the remaining LacZalpha-peptide was used only for transcriptional studies in a suitable host strain (e.g. DHSalpha). The nt sequence of the entire plasmid pDP_mgdf is shown in SEQ ID NO: 28.

Genes dkgA and dkgB encoding multifunctional MG-reductase (SEQ ID NO: 33) and 4-nitrobenzaldehyde reductase (SEQ ID NO: 34), respectively, were amplified from E. coli using Taq-polymerase and the primers dkgB_up and dkg_dw or dkgA_up and dkgA_dw. Purified PCR-products were introduced into vector pCR2.1 (FIG. 3b) by TA-cloning (Invitrogen).

TABLE 1
Primers used for PCR-amplification of genes to be cloned
in pB2araJ or pCR2.1

| | | | SEQ |
|---|---|---|---|
| | | Restriction | ID |
| Primer | Sequence (5′- . . . -3′) | site | NO |
| | for cloning in pB2araJ | | |
| gldH_for1 | GGGGACGTCAAGAAGGAGATATACATATGGACC | AatII | 1 |
| | GCATTATTCAATCACCGG | | |
| gldH_rev1 | GGGACTATTTAAATTATTCCCACTCTTGCAGGAA | SwaI | 2 |
| | ACGC | | |
| dhaK_for1 | GGGACTATTTAAATAAGAAGGAGATATACATATGT | SwaI | 3 |
| | CTCAATTCTTTTTTAACCAAC | | |
| dhaK_rev1 | GGGGCGCGCCTTAGCCCAGCTCACTCTCCGCTA | AscI | 4 |
| | GC | | |
| fucO_for1 | GGGGCCTAGGAAGAAGGAGATATACATATGATGG | AvrII | 5 |
| | CTAACAGAATGATTCTGA | | |
| fucO_rev1 | ACTGCCCGGGCTTACCAGGCGGTATGGTAAAGCT | SmaI | 6 |
| | CT | | |
| mgsA_xhoI—for | TGCTCGAGTAGGCCTAAGAAGGAGATATACATATG | XhoI | 7 |
| | TACATTATGGAACTGACG | | |
| mgsA_xhoI—rev | ATCTCGAGTTACTTCAGACGGTCCGCGA | XhoI | 8 |
| mgsAKO_for | CGCCGATTCCGGTAAAGCTG | — | 9 |
| mgsAKO_rev | GATCCTGGCGCGTTACCATC | — | 10 |
| | for cloning in pCR2.1 | | |
| dkgB_up | TTGGCGCGCCGAATTTAAGGAATAAAGATAATGGC | — | 11 |
| | TATCCCTGCATTTGG | | |
| dkgB_dw | TTGGCGCGCCCTTAATCCCATTCAGGAGCC | — | 12 |
| dkgA_up | TTGGCGCGCCGAATTTAAGGAATAAAGATAATGGC | — | 13 |
| | TAATCCAACCG | | |
| dkgA_dw | TTGGCGCGCCCTTAGCCGCCGAACTGGTCAG | — | 14 |

2.3 Deletion of Activities Encoded by gloA, gloB, rpoS, aldA, ldhA Within E. coli Host Strains

Several techniques for specific gene-deletion are known to those skilled in the art. These techniques comprise, but are not limited to, gene disruption by modified group II introns (Karberg, M., 2001), phage-recombinase mediated gene inactivation using PCR-amplified DNA (Datsenko, K. A., 2000) (Ellis, E. H., 2001) (Yu, D., 2000) (Marx, and Lidstrom, 2002) and introducing linear double stranded DNA homologous to the gene of interest into host cells (Cunningham, R. P., et al. (1980)).

The technique preferentially used to introduce these genes into the strain is electroporation, which is well known to those skilled in the art.

In this invention, homologous recombination of PCR-amplified DNA harbouring selectable marker genes was used. Primers were specifically designed for the genes of interest. Primer sequences are listed below. Successful deletion of the gene of interest was verified by PCR-analysis and DNA-sequencing.

Denotation of deleted genes (1-6) and primers used for generation of homologous linear DNA:

1) Name: subunit of aldehyde dehydrogenase A
Gene: aldA
Accession number: Ecogene: EG10035
Chromosomal localisation: 1486256=>1487695

Primer 1:
(SEQ ID NO: 15)
AACAATGTATTCACCGAAAACAAACATATAAATCACAGGAGTCGCCCATG
Primer 2:
(SEQ ID NO: 16)
GAGGAAAAAACCTCCGCCTCTTTCACTCATTAAGACTGTAAATAAACCAC

2) Name: D-lactate dehydrogenase
Gene: ldhA
Accession number: Ecogene: EG13186
Chromosomal localisation: 1440867=>1439878

Primer 1:
(SEQ ID NO: 17)
CTCCCCTGGAATGCAGGGGAGCGGCAAGATTAAACCAGTTCGTTCGGGCA
Primer 2:
(SEQ ID NO: 18)
TATTTTTAGTAGCTTAAATGTGATTCAACATCACTGGAGAAAGTCTTATG

3) Name: RNA polymerase, sigma S (sigma 38) factor
Gene: rpoS
Accession number: Ecogene: EG10510
Chromosomal localisation: 2865573=>2864581

Primer 1:
(SEQ ID NO: 19)
TGAGACTGGCCTTTCTGACAGATGCTTACTTACTCGCGGAACAGCGCTTC
Primer 2:
(SEQ ID NO: 20)
CTTTTGCTTGAATGTTCCGTCAAGGGATCACGGGTAGGAGCCACCTTATG

### 4) Name: Glyoxylase I

Gene: gloA
Accession number: Ecogene: EG13421
Chromosomal localisation: 1725861=>1726268

Primer 1:
(SEQ ID NO: 21)
TACTAAAACAACATTTTGAATCTGTTAGCCATTTTGAGGATAAAAAGATG
Primer 2:
(SEQ ID NO: 22)
GGCGCGATGAGTTCACGCCCGGCAGGAGATTAGTTGCCCAGACCGCGACC

### 5) Name: Glyoxylase II

Gene: gloB
Accession number: Ecogene: EG13330
Chromosomal localisation: 234782=>234027

Primer 1:
(SEQ ID NO: 23)
CGAACGGAGCCGATGACAAGAAAGTTTTATCAGAACCTATCTTTCTTTGA
Primer 2:
(SEQ ID NO: 24)
CTTGCCGGTTTCATCACAACCTTCCGTTTCACACTGAGAGGTAATCTATG

6) Name: L-ribulokinase monomer
Gene: araB
Accession number: Ecogene: EG10053
Chromosomal localisation: 70048=>68348

Primer 1:
(SEQ ID NO: 25)
AATTATCAAAAATCGTCATTATCGTGTCCTTATAGAGTCGCAACGGCCTG
Primer 2:
(SEQ ID NO: 26)
ACTCTCTACTGTTTCTCCATACCCGTTTTTTTGGATGGAGTGAAACGATG

### Example 3

### 12-PD Production

### 3.1 Culturing of Microorganisms in Crude-Glycerol-Minimal Medium

Utilisation of preparations of crude glycerol (purity about 85%) compared to essentially pure preparations of glycerol (purity>99%) by different microorganisms was investigated. A broad variety of microorganisms representing different taxa along with E. coli MG1655 were grown at unregulated pH in minimal medium supplemented with different amounts of pure and crude glycerol under oxic conditions. Tab. 2 and Tab. 3 demonstrate that no inhibitory effect on biomass production was observed when crude preparation of glycerol instead of pure glycerol was the sole source of carbon and energy. Furthermore, the amount of phosphate in a defined minimal medium can be reduced by 70% when crude glycerol served as source for carbon and energy (Tab. 4).

TABLE 2
Biomass-production sustained by crude-glycerol preparations. Biomass
production was compared when pure (purity >99%) or crude
preparations of glycerol served as carbon source (10 g/l) for growth
under oxic or anoxic condtions. Strains representing different taxa
isolated from environmental samples were cutlivated in microtiterplates
without agitation under atmospheric conditions specified.

| | | biomass production by crude glycerol |
|---|---|---|
| | | equal or higher compared to pure |
| | | preparations of glycerol |
| | Total no. | [%] |
| isolates tested | 374 | — |
| oxic conditions | 304 | 91.3 |
| anoxic condtions | 369 | 98.7 |

TABLE 3
Comparison of biomass-production of E. coli MG1655 obtained by
crude- or pure preparations of glycerol. Biomass production was
compared when pure (purity >99%) or crude preparations of glycerol
served as carbon source for growth under oxic conditions at
unregulated pH. Strains were cutlivated in cultivation tubes under
oxic conditions Ø, average; stdev, standard deviation

| | Biomass production [OD580] | |
|---|---|---|
| substrate | crude glycerol | pure glycerol |

| ]g/l] | Ø | stdv | Ø | stdv |
|---|---|---|---|---|
| 0.63 | 0.3 | 0.09 | 0.5 | 0.09 |
| 1.25 | 0.6 | 0.16 | 0.6 | 0.00 |
| 2.5 | 1.0 | 0.16 | 1.2 | 0.00 |
| 10 | 4.7 | 0.10 | 5.0 | 0.33 |
| 20 | 5.9 | 0.41 | 6.0 | 0.75 |
| 40 | 7.1 | 1.84 | 5.7 | 0.82 |

TABLE 4
Potential of impurities present in crude glycerol (10 g/l) to substitute
for macro-elements of minimal medium. Biomass-production of
E. coli MG1655 was compared when pure (purity >99%)
or crude preparations of glycerol served as source for carbon
and macro-elements for growth under oxic condtions at
unregulated pH. Ø, average; stdev, standard deviation

| | Biomass production [OD580] | |
|---|---|---|
| cultue broth | crude glycerol | pure glycerol |

| devoid of | Ø | stdev | Ø | stdev |
|---|---|---|---|---|
| none | 4.6 | 0.75 | 2.5 | 0.34 |
| nitrogen | 1.0 | 0.00 | 1.1 | 0.09 |
| phosphate | 3.2 | 0.75 | 1.2 | 0.16 |
| sulfate | 1.5 | 0.57 | 1.0 | 0.16 |
| trace-elements | 2.9 | 0.34 | 2.4 | 0.33 |

3.2 Constitution of a Functional Pathway Yielding Dihydroxyacetone Phosphate Bypassing Glycerol Kinase and glyceraldehyde-3-phosphate Dehydrogenase

Genes gldA from E. coli encoding glycerol dehydratase, dhaK from Citrobacter freundii encoding dihydroxyacetone kinase (dhaK) and fucO from E. coli encoding propanediol-oxidoreductase were cloned in plasmid pB2araJ to give plasmid pDP_gdf. Plasmid pDP_mgdf additionally contains methylglyoxal synthase from E. coli. According to known biochemical pathways, propanediol oxidoreductase is not relevant for this example. However, we tested plasmid pDP_gdf and its derivative, pDP_mgdf for functional complementation of a glpK-knock out, since these plasmids are relevant for recombinant 1,2-PD production.

The assay demonstrated that the presence of plasmid pDP_gdf or pDP_mgdf relieved inhibition of growth in a glpK-mutant of E. coli when glycerol was the sole carbon source (Tab. 5). Thus, an inducible unregulated pathway independent of the endogenous route to metabolise glycerol was established. The biomass finally obtained is equal or even higher compared to growth of a control strain (DH5alpha□araB) or when glucose is the substrate for growth.

TABLE 5
An araB-knockout of E. coli DH5alpha and derivatives
thereof were cultivated in minimal medium containing
glucose or glycerol as carbon source at 10 g/l.

| | | | | Biomass production | | | |
|---|---|---|---|---|---|---|---|
| | | | | Carbon source | | | |
| E. coli | Genes | | Plasmid | Glucose | | Glycerol | |
| strain | inacitvated | | pDP_{—} | OD580 | stdev | OD580 | stdev |
| DH5alpha | araB | — | — | 3.7 | — | 2.0 | 0.1 |
| DH5alpha | araB | glpK | — | 3.0 | — | 0.1 | 0.0 |
| DH5alpha | araB | glpK | gdf | 2.7 | — | 4.5 | 0.2 |
| DH5alpha | araB | glpK | mgdf | 5.6 | 0.2 | 5.5 | 0.3 |

The different strains were cultivated at 37° C. in cultivation tubes under oxic conditions for 5 days. Biomass was determined as optical density at 580 nm at the end of the expriment. Stdev, standard deviation

3.3 Defining a Minimal Set of Genes Indispensible for Recombinant 1,2-PD Production from Glycerol

An araB mutant of E. coli DHSalpha was transformed with different plasmids containing genes of interest listed in Table 6. The wild-type (control) and recombinant strains were cultivated under oxic conditions in minimal medium containing 10 g/l crude glycerol for 3-5 days until the strains entered stationary phase. Supernatants were analysed by GC-MS analysis for 1,2-PD contents. Results are given in table 6.

TABLE 6
1,2-PD contents determined in supernatants upon cultivation of E. coli
strains in minimal medium containing 10 g/l of crude glycerol.

| E. coli | genes | | genes | 1,2-PD | stdev |
|---|---|---|---|---|---|
| strain | inactivated | plasmids | expressed | [mg/l] | [mg/l] |
| DH5a | araB | none | — | 0 | 0 |
| DH5a | araB | pB2araJ | — | 0 | 0 |
| DH5a | araB | pDP_g | gldA | 0 | 0 |
| DH5a | araB | pDP_f | fucO | 39 | 4 |
| DH5a | araB | pDP_gd | gldA, dhaK | 0 | 0 |
| DH5a | araB | pDP_gd | gldA, dhaK, | 0 | 0 |
| | | pUC_mgsA | mgsA | | |
| DH5a | araB | pDP_gdf | gldA, dhaK, fucO | 40 | 3 |
| DH5a | araB | pDP_gdf | gldA, dhaK, | 59 | 9 |
| | | pUC_mgsA | fucO, mgsA | | |

3.4 Exclusive Production of the 1,2-isomer of Propanediol by Recombinant E. coli Strains Expressing Glycerol Dehydrogenase, Dihydroxyacetone Kinase, Methylglyoxal Synthase and Propanediol Oxidoreductase Encoded in a Synthetic Operon

E. coli MG1655□araB was transformed with or without plasmid pDP_mgdf and cultivated in minimal-medium containing 10 or 15 g/l crude glycerol as carbon source. Incubation was done under oxic conditions in cultivation tubes, or in sealed glass vials without agitation (semi-anoxic conditions). E. coli MG1655□araB without plasmid was the control strain. Incubation period was 5 days, incubation temperature was 37° C.

At the end of the experiment, growth was determined as optical density (OD580), and 1,2- or 1,3-PD levels were determined by GC-MS analysis. Assays were done in duplicate. The results (Tab. 7) demonstrate successful introduction of an engineered pathway that enables E. coli to produce 1,2-PD from crude glycerol that produces exclusively the 1,2-isomer of propanediol.

TABLE 7
araB-knock out stains of E. coli MG1655 were transformed with/without
plasmid pDP_mgdf and cultivated in minimal medium containing crude
glycerol as carbon source at concentrations of 10 or 15 g/l.

| | | | | substrate | propanediol production | | | |
|---|---|---|---|---|---|---|---|---|
| E. coli | genes | | | crude | 1,2-PD [mg/l] | | 1,3-PD [mg/1] | |
| strain | inactivated | plasmids | cultivation | glycerol [g/l] | Ø | stdev | Ø | stdev |
| MG1655 | araB | none | oxic | 10 | 0 | 0 | 0 | 0 |
| MG1655 | araB | none | oxic | 15 | 0 | 0 | 0 | 0 |
| MG1655 | araB | pDP_mgdf | oxic | 10 | 203 | 5 | 0 | 0 |
| MG1655 | araB | pDP_mgdf | oxic | 15 | 435 | 11 | 0 | 0 |

Cultivation was done at 37° C. under oxic and semi-anoxic conditions. Biomass was determined as optical density at 580 nm at the end of the experiment (5 days); 1,2-PD contents in the supernatant were determined by GC-MS analysis; 0, not detected, detection-limit 10 mg/l. Ø, average; stdev, standard deviation

3.5 Toxicity of Methyglyoxal to Wild-Type and Mutant Strains of E. coli

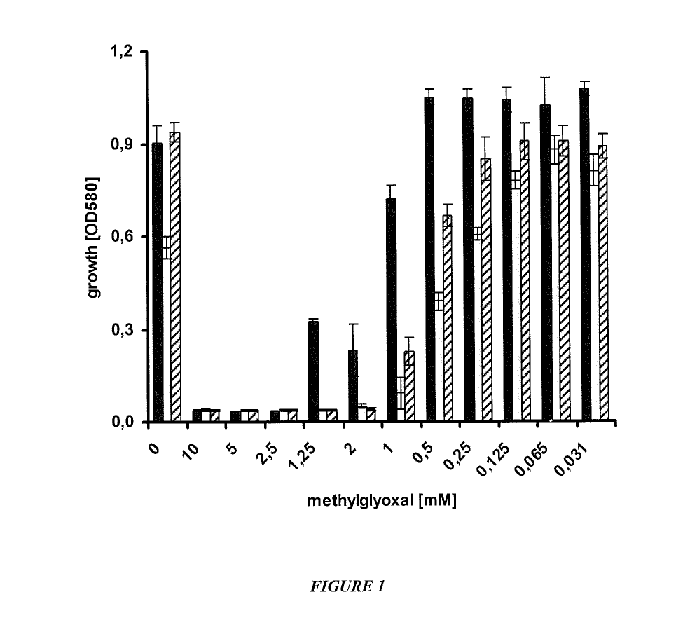

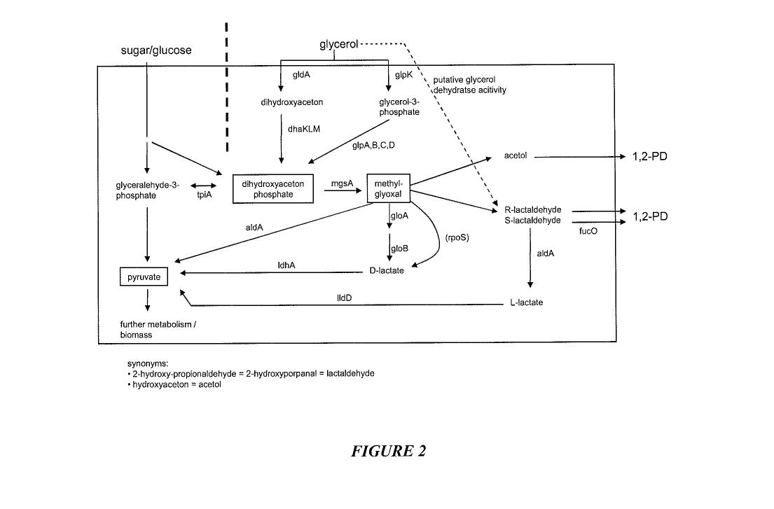

E. coli MG1655 wild-type or mutants inactivated in the genes gloA and gloB, respectively, were cultivated in minimal-medium with pure-glycerol (10 g/l) as source for carbon and energy containing different amounts of methylglyoxal. Tests for methylglyoxal indicated a period of at least 72 h of chemical stability. E. coli was cultivated in microtiterplates without agitation at 37° C. under a humid atmosphere. Growth was determined at OD580 48.5 h after inoculation (FIG. 1; black bars, E. coli MG1655 wild-type; grey bars, E. coli MG1655 gloA-mutant; hatched bars, E. coli MG1655 gloB-mutant). Inhibition of growth of the wild-type strain was observed for extracellular concentration of methylglyoxal equal or higher than 2.5 mM. The mutant strains were significantly more sensitive, especially the glyoxylase I mutant (gloA), thus substantiating our finding, that gloA is a major drainage pathway for intracellular methylglyoxal (see 4.6). The results further demonstrate that elevated levels of methylglyoxal inhibit growth of E. coli which is a basis for the identification of phosphate-insensitive variants of methylglyoxal-synthases.

### 3.6 Identification of Major MG-Withdrawing Activity

E. coli MG1655 was inactivated (gene knockout) in the genes whose gene products are involved in methylglyoxal (MG) metabolism, e.g. detoxification of MG. The genes are listed in table 8. Wild-type and mutant strains were cultivated in cultivation tubes under oxic conditions and agitation until growth ceased. Levels of 1,2-PD in the bulk liquid were determined by GC-MS analysis and compared to levels that were found when glucose (negative control) was source for carbon and energy. When glycerol was substrate for growth, a background level of about 20 mg/ml 1,2-PD was detected. However, significantly elevated 1,2-PD levels were observed for the gloA-mutant, solely.

TABLE 8
Strains of E. coli MG1655 mutated in genes involved
in methylglyoxal metabolism were cultivated in minimal
medium containing glycerol or glucose (10 g/l).

| | | 1,2-PD | | 1,3-PD | | | |
|---|---|---|---|---|---|---|---|
| E. coli | gene(s) | [mg/l] | | [mg/l] | | OD580 | |
| strain | inactivated | Ø | stdev | Ø | stdev | Ø | stdev |
| MG 1655 | none | 20 | 1 | 0 | 0 | 2.6 | 0.2 |
| MG 1655 | none | 0 | 0 | 0 | 0 | 2.1 | 0.1 |
| MG 1655 | gloB | 33 | 1 | 0 | 0 | 2.6 | 0.2 |
| MG 1655 | gloB | 0 | 0 | 0 | 0 | 2.1 | 0.1 |
| MG 1655 | ldhA | 22 | 1 | 0 | 0 | 2.7 | 0.3 |
| MG 1655 | ldhA | 0 | 0 | 0 | 0 | 2.2 | 0.3 |
| MG 1655 | rpoS | 24 | 0 | 0 | 0 | 2.7 | 0.4 |
| MG 1655 | rpoS | 0 | 0 | 0 | 0 | 1.9 | 0.1 |
| MG 1655 | gloA | 113 | 5 | 0 | 0 | 2.6 | 0.2 |
| MG 1655 | gloA | 3 | 5 | 0 | 0 | 1.9 | 0.2 |
| MG 1655 | aldA | 25 | 1 | 0 | 0 | 2.7 | 0.1 |
| MG 1655 | aldA | 0 | 0 | 0 | 0 | 2.3 | 0.1 |
| MG 1655 | aldA, gloA | 102 | 6 | 0 | 0 | 1.6 | 0.0 |
| MG 1655 | aldA, gloA | 0 | 0 | 0 | 0 | 1.8 | 0.3 |
| MG 1655 | aldA, ldhA, gloA | 79 | 9 | 0 | 0 | 2.7 | 0.3 |
| MG 1655 | aldA, ldhA, gloA | 0 | 0 | 0 | 0 | 2.5 | 0.3 |
| MG 1655 | aldA, rpoS, gloA | 110 | 0 | 0 | 0 | 2.8 | 0.0 |
| MG 1655 | aldA, rpoS, gloA | 0 | 0 | 0 | 0 | 2.5 | 0.8 |

Cultivation was performed under oxic conditions at 37° C. until growth ceased. Growth was determined as optical density (OD580); Propanediol levels were determined by GC-MS analysis. Ø, average; stdev, standard deviation

### 3.7 Recombinant Organisms Producing High Levels of 1,2-PD

E. coli MG1655 was inactivated (gene knockout) in the genes araB, or in genes aldA and gloA. Corresponding mutants were transformed with plasmid pDP_mgdf. AraB-mutants harbouring plasmid pDP_mgdf were additionally transformed with plasmid pCR2.1 encoding genes conferring aldo-keto reductase activity, e.g. dkgA or dkgB of E. coli. Cells were cultivated in presence of crude-glycerol (15 g/l) under oxic or semi-anoxic conditions at 37° C. for 2-5 days or until optical density failed to increase further. Supernatants were analysed for 1,2-PD content by GC-MS analysis (Tab. 9).

TABLE 9
Mutant strains of E. coli MG1655 expressing glycerol-to-1,2-PD converting genes
were cultivated in minimal medium containing glycerol or glucose (15 g/l).

| E. coli | gene(s) | plasmids present | | | 1,2-PD [mg/l] | | 1,3-PD [mg/l] | |
|---|---|---|---|---|---|---|---|---|
| strain | inactivated | plasmid 1 | plasmid 2 | cultivation | Ø | stdev | Ø | stdev |
| MG 1655 | aldA, gloA | none | none | oxic | 65 | 5 | 0 | 0 |
| MG 1655 | aldA, gloA | none | none | semi-anoxic | 30 | 0 | 0 | 0 |
| MG 1655 | aldA, gloA | pDP_mgdf | none | oxic | 340 | 60 | 0 | 0 |
| MG 1655 | aldA, gloA | pDP_mgdf | none | semi-anoxic | 370 | 20 | 0 | 0 |
| MG 1655 | araB | pDP_mgdf | pCR_dkgA | oxic | 440 | 110 | 0 | 0 |
| MG 1655 | araB | pDP_mgdf | pCR_dkgA | semi-anoxic | 260 | 0 | 0 | 0 |
| MG 1655 | araB | pDP_mgdf | pCR_dkgB | oxic | 275 | 15 | 0 | 0 |
| MG 1655 | araB | pDP_mgdf | pCR_dkgB | semi-anoxic | 340 | 170 | 0 | 0 |

Cultivation was performed under oxic or semi-anoxic conditions at 37° C. until growth ceased. Propanediol levels in supernatants were determined by GC-MS analysis. Ø, average; stdev, standard deviation

### Results

As shown in table 2 and 3, crude preparations of glycerol can be utilised without further processing by a wide variety of organisms not restricted to E. coli and close relatives. Biomass production obtained by utilizing crude glycerol is equal to or higher when compared with pure glycerol as carbon substrate. Thus, crude-glycerol substitutes for pure preparations of glycerol in virtually any process that is based on the fermentation of glycerol.

As shown in table 4, impurities present in crude-glycerol provide a source of phosphor sustaining substantial growth of host cells. Thus, the amount of phosphor added to the culture broth can be decreased by about 70%.

As shown in table 6, propanediol oxidoreductase activity is indispensible for the synthesis of 1,2-PD from glycerol. Host cells that express glycerol-dehydrogenase do not produce 1,2-PD. Cells that express glycerol-dehydrogenase and methylglyoxal-synthase but lacking propanediol-oxidoreductase do not produce detectable amounts of 1,2-PD, whereas additional presence of propanediol-oxidoreductase activity results in significant 1,2-PD production.

As shown in table 7, high titers of 1,2-PD are obtained from glycerol with recombinant strains coexpressing methylglyoxal-synthase, glycerol-dehydrogenase, dihydroxyacetone-kinase and propanediol-oxidoreductase under oxic conditions. The synthesis of propanediol based on the present invention results in the synthesis of exclusively the 1,2-isomer but not the 1,3-isomer of propanediol.

As shown in table 8, inactivation of glyoxylase I activity encoded by gloA in E. coli results in significant production of 1,2-PD from glycerol. Thus, enzyme activity encoded by gloA is the major competing activity interfering with high-level production of 1,2-PD in host ells.

### EMBODIMENTS OF THE INVENTION

1. A host cell engineered to produce high levels of 1,2-propanediol when grown on glycerol as the sole carbon source.
2. A host cell according to the preceding embodiment, wherein the glycerol has a degree of purity of at least 70%, particularly of at least 75%, particularly of at least 80%, particularly of at least 85%, particularly of at least 90%, particularly of at least 95%, particularly of at least 99% and up to 100%.
3. A host cell according to any of the preceding embodiments, wherein the glycerol has a degree of purity of between 80% and 90%.
4. A host cell according to any of the preceding embodiments, wherein the glycerol has a degree of purity of about 85%.
5. A host cell according to any of the preceding embodiments, wherein the glycerol is a crude glycerol preparation from biodiesel and/or bioethanol production.
6. A host cell according to any of the preceding embodiments wherein said host cell has been engineered through recombinant DNA techniques.
7. A host cell according to any of the preceding embodiments, particularly to embodiment 6, wherein said host cell has been engineered by introducing a gene encoding a propanediol oxidoreductase activity (fucO).
8. A host cell according to embodiment 7, wherein said host cell has been engineered by introducing at least one additional gene encoding an enzyme activity selected from the group consisting of glycerol dehydrogenase (gldA), dihydroxyacetone kinase (dhaK) and methylglyoxalsynthase (mgsA) such as to express said activities along with the propanediol oxidoreductase activity (fucO).
9. A host cell according to embodiment 7, wherein said host cell has been engineered by introducing an additional gene encoding a glycerol dehydrogenase such as to express said glycerol dehydrogenase activity along with the propanediol oxidoreductase activity.
10. A host cell according to embodiment 7, wherein said host cell has been engineered by introducing an additional gene encoding a dihydroxyacetone kinase such as to express said dihydroxyacetone kinase activity along with the propanediol oxidoreductase activity.
11. A host cell according to embodiment 7, wherein said host cell has been engineered by introducing an additional gene encoding a methylglyoxalsynthase (mgsA) such as to express said methylglyoxalsynthase activity along with the propanediol oxidoreductase activity.
12. A host cell according to embodiment 7, wherein said host cell has been engineered by introducing additional genes encoding a glycerol dehydrogenase and a dihydroxyacetone kinase such as to express said glycerol dehydrogenase and dihydroxyacetone kinase activities along with the propanediol oxidoreductase activity.
13. A host cell according to embodiment 7, wherein said host cell has been engineered by introducing additional genes encoding a glycerol dehydrogenase and a methylglyoxalsynthase such as to express said glycerol dehydrogenase and methylglyoxalsynthase activities along with the propanediol oxidoreductase activity.
14. A host cell according to embodiment 7, wherein said host cell has been engineered by introducing additional genes encoding a dihydroxyacetone kinase and a methylglyoxalsynthase such as to express said dihydroxyacetone kinase and methylglyoxalsynthase activities along with the propanediol oxidoreductase activity.
15. A host cell according to embodiment 7, wherein said host cell has been engineered by introducing additional genes encoding a glycerol dehydrogenase, a dihydroxyacetone kinase and a methylglyoxalsynthase such as to express said glycerol dehydrogenase, dihydroxyacetone kinase and methylglyoxalsynthase activities along with the propanediol oxidoreductase activity.
16. A host cell according to any of the preceding embodiments, particularly to embodiment 7, wherein said host cell has been engineered by introducing additional genes encoding a glycerol dehydratase such as to express said glycerol dehydratase activity along with the propanediol oxidoreductase activity.
17. A host cell according to any of the preceding embodiments, particularly to embodiment 7, wherein said host cell has been engineered by introducing additional genes encoding an aldo-keto-reductase such as to express said aldo-keto-reductase activity along with the propanediol oxidoreductase activity.
18. A host cell according to embodiment 17, wherein said aldo-keto-reductase activity is contributed by a gene selected from the group consisting of dkgA, dkgB, yeaE and yghZ.
19. A host cell according to any of the preceding embodiments, wherein said host cell is defective in arabinose metabolism.
20. A host cell according to the preceding embodiment, wherein said defect is due to a reduced or missing ribulose kinase activity.
21. A host cell according to any of the preceding embodiments, wherein said host cell is defective in the metabolism of methylglyoxal.
22. A host cell according to the preceding embodiment, wherein said defect is due to a reduced or missing enzyme activity selected from the group consisting of glyoxylase system I, glyoxylase system II, lactate dehydrogenase A, glyoxylase system III, aldehyde dehydrogenase A activity, but particularly a glyoxylase system I activity.
23. A host cell according to any of the preceding embodiments, wherein said host cell is defective in the metabolism of dihydroxyacetonphosphate.
24. A host cell according to the preceding embodiment, wherein said defect is due to a reduced or missing triosephosphate isomerase activity.
25. A host cell according to any of the preceding embodiments, which produces high levels of 1,2-propanediol when grown on glycerol as the sole carbon source, but essentially no 1,3-propanediol.
26. A host cell according to any of the preceding embodiments, which is a microbial or a fungal host cell.
27. A microbial host cell according to embodiment 26, which is E. coli.
28. A polynucleotide molecule comprising a synthetic operon under the control of an inducible promoter, which operon comprises the genes encoding glycerol dehydrogenase (gldA) and propanediol oxidoreductase (fucO).
29. A polynucleotide molecule comprising a synthetic operon under the control of an inducible promoter, which operon comprises the genes encoding glycerol dehydrogenase (gldA), dihydroxyacetone kinase (dhaK) and propanediol oxidoreductase (fucO).
30. A polynucleotide according to embodiment 29 wherein the synthetic operon is extended to further contain a gene encoding methylglyoxal synthase (mgsA).
31. A polynucleotide according to embodiment 29 and 30, wherein the genes encoding glycerol dehydrogenase (gldA); propanediol oxidoreductase (fucO) and methylglyoxal synthase (mgsA), respectively, are obtainable from E. coli and the gene encoding dihydroxyacetone kinase (dhaK) is obtainable from C. freundii.
32. A polynucleotide according to any of the preceding embodiments, which is a plasmid.
33. A polynucleotide according to any of the preceding embodiments wherein the arrangement of the genes in the synthetic operon is 5′-mgsA-gldA-dahK-fucO-3′.
34. A polynucleotide according to any of the preceding embodiments wherein the inducible promoter is an arabinose-inducible promoter.
35. A microorganism according to any of the preceding embodiments comprising a polynucleotide according to anyone of embodiments 28 to 34.
36. A microorganism according to any of the preceding embodiments comprising a phosphate-insentive mgsA gene, which is fully operable under high phosphate concentrations, particularly under phosphate concentrations higher than 0.7 mM in the cultivation medium or higher than 9,3e-05 in the cytoplasm of the cell.
37. A method for the production of 1,2-propanediol comprising growing a host cell according to any one of embodiments 1 to 27 in an appropriate growth medium containing a simple carbon source, particularly a crude glycerol preparation, after which the 1,2-propanediol produced are recovered and, optionally, purified.
38. A method according to embodiment 37, comprising:
   i) culturing a host cell according to any one of embodiments 1 to 27, which host cell overexpresses propanediol oxidoreductase (fucO) activity, in a medium containing a non-fermentable carbon substrate, whereby the carbon substrate is sustaining production of biomass and serves as a substrate for production of 1,2 propanediol (1,2-PD) at the same time, and the non-fermentable carbon source is metabolized by the host cell into 1,2-propanediol;
   ii) recovering the 1,2-propanediol produced according to step i); and, optionally,
   iii) purifying the recovered 1,2-propanediol.
39. A method according to any of the preceding embodiments, wherein said non-fermentable carbon substrate is a crude glycerol preparation, particularly a preparation containing glycerol with a purity of at least 70%, particularly of at least 75%, particularly of at least 80%, particularly of at least 85%, particularly of at least 90%, particularly of at least 95%, particularly of at least 99% and up to 100%.
40. A method according to any of the preceding embodiments, wherein the glycerol has a degree of purity of between 80% and 90%, particularly of about 85%.
41. A method according to any of the preceding embodiments, wherein the non-fermentable carbon substrate, particularly the crude glycerol preparation is selectively metabolized to 1,2-propanediol.
42. A method according to any of the preceding embodiments, wherein a host cell is used, which is engineered to overexpress propanediol oxidoreductase (fucO).
43. A method according to any of the preceding embodiments, wherein a host cell is used, which is engineered to co-express at least one enzyme selected from the group consisting of glycerol dehydrogenase (gldA), dihydroxyacetone kinase (dhaK) and methylglyoxalsynthase (mgsA) along with the propanediol oxidoreductase (fucO) activity.
44. A method according to any of the preceding embodiments, wherein a host cell is used, wherein at least one enzyme activity involved in a non-productive pathway competing with 1,2-PD production has been deactivated.
45. A method according to any of the preceding embodiments, wherein a microbial mutant, particularly a mutant of E. coli, is used, where one or more of the genes encoding glyoxylase systems I and II (gloA and gloB), lactate dehydrogenase A (ldhA), glyoxylase system III (indirectly by inactivation of the master regulator rpoS), and aldehyde dehydrogenase have been deactivated.
46. A method according to any of the preceding embodiments, wherein a microbial mutant or strain, particularly an E. coli mutant, is used where the gene encoding a gloA activity has been partially or fully inactivated:
47. A method according to any of the preceding embodiments, wherein a microbial mutant or strain inactivated in arabinose metabolism is used.
48. A method according to any of the preceding embodiments, wherein an E. coli strain is used as the host organism, particularly an E. coli strain MG1655 and DHSalpha, respectively.
49. A method according to any of the preceding embodiments, wherein at least one of the genes encoding an enzyme activity selected from the group consisting of glycerol dehydrogenase (gldA), dihydroxyacetone kinase (dhaK) and methylglyoxalsynthase (mgsA) and propanediol oxidoreductase (fucO) is under the control of an inducible promoter, particularly an arabinose inducible promoter, particularly a paraBAD promoter.
50. A method according to any of the preceding embodiments, wherein a synthetic operon is used in the method according to the invention to provide a host cell co-expressing at least one enzyme activity selected from the group consisting of glycerol dehydrogenase (gldA), dihydroxyacetone kinase (dhaK) and methylglyoxalsynthase (mgsA) activity along with the propanediol oxidoreductase (fucO) activity.
51. A method according to any of the preceding embodiments, wherein the genes encoding the above activities are under control of an inducible promoter, particularly an arabinose-inducible promoter, but especially a paraBAD promoter.
52. A method according to any of the preceding embodiments, wherein the succession of genes transcribed upon induction from said operon is as follows: mgsA, gldA, dhaK, fucO.
53. A method for the preparation of a host cell that can be used in a method according to any one embodiments 37 to 52 for the production of 1,2-propanediol comprising transforming said host cell with a polynucleotide according to any one of embodiments 27 to 33.
54. A method according to embodiment 53, wherein said host cell is a microbial host cell, particularly E. coli.
55. A method according to embodiment 53, wherein transformation is accomplished by electroporation.
56. A host cell produced by a method according to any one of embodiments 53 to 55.

### REFERENCE LIST

Altaras, N. E, 2001; Biotechnology Progress (17), 52-56
Appleyard, R. K., Genetics, 39, 440-452 (1954)
Ausubel, Current Protocols in Molecular Biology, Green Publishing Associates and Wiley Interscience, N.Y. (1994).
Ausubel, F. M., Brent, R., Kingston, R. E., Moore, D. D., Seidman, J. G., Smith, J. A. & Struhl, K. (1995). Short Protocols in Molecular Biology. A Compendium of Methods from Current Protocols in Molecular Biology, 3 edn. New York: John Wiley & Sons, Inc.
Balzer, et al., Nucleic Acid Res. 19:5081 (1991);
Cameron, D. C., 1986; Bio/Technology (4), 651-654
Cameron, D. C, 1998; Biotechnology Progress (14), 116-125
Cunningham, R. P., DasGupta, C., Shibata, T., and Radding, C. M. (1980) Cell 20, 223-235).
Datsenko, K. A., 2000; PNAS (97), 6640-6645
Ellis, E. H., 2001; PNAS (98), 6742-6746
Gough J A, Murray N E., J. Mol. Biol., 166, 1-19 (1983)
Grandison, Alistair S., Sep. Processes Food Biotechnol. Ind. (1996), 155 177.
Guzman, L. M., 1995; Journal of Bacteriology (177), 4121-4130
Hanahan J., et al., Mol. Biol., 166, 557-580 (1983)
Heath, E. C., 1962; The Journal of Biological Chemistry (237), 2423-2426
Hopper, D. J. 1972; Biochemical Journal (128), 321-329
Jones, L. R. & Riddick, J. A. (1957). Colorimetric determination of 1,2-propanediol and related compounds. Anal Chem 39, 1214-1216.
Joyner, A. 1999, Gene Targeting, A Practical Approach, Oxford University Press. ISBN-13: 978-0-19-963792-8.
Karberg, M., 2001; Nature Biotechnology (19), 1162-1167
Kluyver and Schellen, 1937
Le Mouellic, K. et al, Proc. Natl. Acad. Sci. USA, 87 (1990), 4712-4716
Marx, C. J. and Lidstrom, M. E., (2002), BioTechniques (33)₅, 1062-1067
Miller, J. H. (1992). A Short Course in Bacterial Genetics: A Laboratory Manual and Handbook for Escherichia Coli and Related Bacteria, 1st edn: Cold Spring Harbor Laboratory Press.
Neidhardt et al. 1996, Escherichia coli and Salmonella: Cellular and Molecular Biology, ASM Press
Ohtsuka, et al., J. Biol. Chem. 260:2605-2608 (1985)
Pfennig, N. & Lippert, K. D. (1966). Über das Vitamin B12-Bedürfnis phototropher Schwefelbakterien. Archives of microbiology 55, 245-256.
Reynolds, A. B. et al. Cell 38, 275-285 (1984).
Rossolini, et al., Mol. Cell. Probes 8:91-98 (1994)
Sambrook et al., Molecular Cloning: A Laboratory Manual, Second Edition (1989) Cold Spring Harbor Laboratory Press, Cold Spring Harbor, N.Y. (1989)
Sambrook, J. & Russell, D. W. (2001). Molecular Cloning: A Laboratory Manual, 3rd edn: Cold Spring Harbor Laboratory Press.
Tran Din, K, 1985; Archives of Microbiology (142), 87-92
Wood, W. B., J. Mol. Biol., 16, 118-133 (1966)
Yu, D., 2000; PNAS (97), 5978-5983
Jerpseth, B. et al. Strategies, 5, 81-83 (1992)
Young, R. A. and R. W. Davis, Science, 222, 778-782 (1983)

### Patent Literature

U.S. Pat. No. 5,008,473 U.S. Pat. No. 4,683,202
U.S. Pat. No. 5,356,812 US Pat. Appl. 2007/072279
U.S. Pat. No. 7,049,109 WO 2005/073364
U.S. Pat. No. 6,087,140
U.S. Pat. No. 6,303,352

What is claimed is:

1. A host cell engineered to produce 1,2-propanediol when grown on glycerol as the sole carbon source.

2. A host cell according to claim 1, wherein the glycerol has a degree of purity between 80% and 90%.

3. A host cell according to claim 1, wherein said host cell has been engineered by introducing a gene encoding a propanediol oxidoreductase activity.

4. A host cell according to claim 3, wherein said host cell has been engineered by introducing at least one additional gene encoding an enzyme activity selected from the group consisting of glycerol dehydrogenase (gldA), dihydroxyacetone kinase (dhaK) and methylglyoxalsynthase (mgsA) such as to express said activities along with the propanediol oxidoreductase activity.

5. A host cell to claim 3, wherein said host cell has been engineered by introducing additional genes encoding a glycerol dehydrogenase, a dihydroxyacetone kinase and a methylglyoxalsynthase such as to express said glycerol dehydrogenase, dihydroxyacetone kinase and methylglyoxalsynthase activities along with the propanediol oxidoreductase activity.

6. A host cell to claim 3, wherein said host cell has been engineered by introducing additional genes encoding a glycerol dehydratase such as to express said glycerol dehydratase activity along with the propanediol oxidoreductase activity.

7. A host cell according to claim 3, wherein said host cell has been engineered by introducing additional genes encoding an aldo-keto-reductase such as to express said aldo-keto-reductase activity along with the propanediol oxidoreductase activity.

8. A host cell according to claim 1, wherein said host cell is defective in at least the metabolism of compounds selected from the group consisting of:
i) arabinose
ii) methylglyoxal
iii) dihydroxyacetonphosphate.

9. A host cell according to claim 1, which produces 1,2-propanediol when grown on glycerol as the sole carbon source, but essentially no 1,3-propanediol.

10. A host cell according to claim 9, which is E. coli.

11. A method for the production of 1,2-propanediol, comprising:
growing a host cell engineered to produce 1,2-propanediol, in an appropriate growth medium containing glycerol as the sole carbon source; and
recovering 1,2-propanediol produced by said host cell.

12. A method according to claim 11 and further including purifying said 1,2-propanediol.

13. A method according to claim 11, wherein said glycerol has a degree of purity between 80% and 90%.

14. A method according to claim 11, wherein said host cell has been engineered by introducing a gene encoding a propanediol oxidoreductase activity.

15. A method according to claim 14, wherein said host cell has been engineered by introducing at least one additional gene encoding an enzyme activity selected from the group consisting of glycerol dehydrogenase (gldA), dihydroxyacetone kinase (dhaK) and methylglyoxalsynthase (mgsA) such as to express said activities along with the propanediol oxidoreductase activity.

16. A method according to claim 14, wherein said host cell has been engineered by introducing additional genes encoding a glycerol dehydrogenase, a dihydroxyacetone kinase and a methylglyoxalsynthase such as to express said glycerol dehydrogenase, dihydroxyacetone kinase and methylglyoxalsynthase activities along with the propanediol oxidoreductase activity.

17. A method according to claim 14, wherein said host cell has been engineered by introducing additional genes encoding a glycerol dehydratase such as to express said glycerol dehydratase activity along with the propanediol oxidoreductase activity.

18. A method according to claim 14, wherein said host cell has been engineered by introducing additional genes encoding an aldo-keto-reductase such as to express said aldo-keto-reductase activity along with the propanediol oxidoreductase activity.

19. A method according to claim 11, wherein said host cell is defective in at least the metabolism of compounds selected from the group consisting of:
i) arabinose
ii) methylglyoxal
iii) dihydroxyacetonphosphate.

20. A method according to claim 11, wherein said host cell produces essentially no 1,3-propanediol.

21. A method according to claim 20, wherein said host cell is E. coli.


SEQUENCE LISTING (34 sequences)

SEQ ID NO: 1 - DNA, 51 bp, Artificial
  primer gldH-for1
ggggacgtcaagaaggagatatacatatggaccgcattattcaatcaccgg

SEQ ID NO: 2 - DNA, 38 bp, Artificial
  primer gldH-rev1
gggactatttaaattattcccactcttgcaggaaacgc

SEQ ID NO: 3 - DNA, 56 bp, Artificial
  primer dhaK-for1
gggactatttaaataagaaggagatatacatatgtctcaattcttttttaaccaac

SEQ ID NO: 4 - DNA, 35 bp, Artificial
  primer dhaK_rev1
ggggcgcgccttagcccagctcactctccgctagc

SEQ ID NO: 5 - DNA, 52 bp, Artificial
  primer fucO-for1
ggggcctaggaagaaggagatatacatatgatggctaacagaatgattctga

SEQ ID NO: 6 - DNA, 36 bp, Artificial
  primer fucO_rev1
actgcccgggcttaccaggcggtatggtaaagctct

SEQ ID NO: 7 - DNA, 53 bp, Artificial
  primer mgsA_xhol_for
tgctcgagtaggcctaagaaggagatatacatatgtacattatggaactgacg

SEQ ID NO: 8 - DNA, 28 bp, Artificial
  primer mgsA_xhol_rev
atctcgagttacttcagacggtccgcga

SEQ ID NO: 9 - DNA, 20 bp, Artificial
  primer mgsAKO_for
cgccgattccggtaaagctg

SEQ ID NO: 10 - DNA, 20 bp, Artificial
  primer mgsAKO_rev
gatcctggcgcgttaccatc

SEQ ID NO: 11 - DNA, 50 bp, Artificial
  primer dkgB_up
ttggcgcgccgaatttaaggaataaagataatggctatccctgcatttgg

SEQ ID NO: 12 - DNA, 30 bp, Artificial
  primer dkgB_dw
ttggcgcgcccttaatcccattcaggagcc

SEQ ID NO: 13 - DNA, 46 bp, Artificial
  primer dkgA_up
ttggcgcgccgaatttaaggaataaagataatggctaatccaaccg

SEQ ID NO: 14 - DNA, 31 bp, Artificial
  primer dkgA_dw
ttggcgcgcccttagccgccgaactggtcag

SEQ ID NO: 15 - DNA, 50 bp, Artificial
  Primer 1_aldA
aacaatgtattcaccgaaaacaaacatataaatcacaggagtcgcccatg

SEQ ID NO: 16 - DNA, 50 bp, Artificial
  Primer 2_aldA
gaggaaaaaacctccgcctctttcactcattaagactgtaaataaaccac

SEQ ID NO: 17 - DNA, 50 bp, Artificial
  Primer 1_ldhA
ctcccctggaatgcaggggagcggcaagattaaaccagttcgttcgggca

SEQ ID NO: 18 - DNA, 50 bp, Artificial
  Primer 2_ldhA
tatttttagtagcttaaatgtgattcaacatcactggagaaagtcttatg

SEQ ID NO: 19 - DNA, 50 bp, Artificial
  Primer 1_rpoS
tgagactggcctttctgacagatgcttacttactcgcggaacagcgcttc

SEQ ID NO: 20 - DNA, 50 bp, Artificial
  Primer 2_rpoS
cttttgcttgaatgttccgtcaagggatcacgggtaggagccaccttatg

SEQ ID NO: 21 - DNA, 50 bp, Artificial
  Primer 1_gloA
tactaaaacaacattttgaatctgttagccattttgaggataaaaagatg

SEQ ID NO: 22 - DNA, 50 bp, Artificial
  Primer 2_gloA
ggcgcgatgagttcacgcccggcaggagattagttgcccagaccgcgacc

SEQ ID NO: 23 - DNA, 50 bp, Artificial
  Primer 1_gloB
cgaacggagccgatgacaagaaagttttatcagaacctatctttctttga

SEQ ID NO: 24 - DNA, 50 bp, Artificial
  Primer 2_gloB
cttgccggtttcatcacaaccttccgtttcacactgagaggtaatctatg

SEQ ID NO: 25 - DNA, 50 bp, Artificial
  Primer 1_araB
aattatcaaaaatcgtcattatcgtgtccttatagagtcgcaacggcctg

SEQ ID NO: 26 - DNA, 50 bp, Artificial
  Primer 2_araB
actctctactgtttctccatacccgtttttttggatggagtgaaacgatg

SEQ ID NO: 27 - DNA, 1659 bp, Citrobacter freundii
atgtctcaattcttttttaaccaacgcacccatcttgtgagcgacgtcatcgacggggcgattatcgccagcccatggaataacctggcgcgtctggaaagcgatccggccattcgcatcgtggtccgtcgtgaccttaataaaaataacgtagcggtcatttccggcggcggttcgggacacgaacccgcgcacgttgggtttatcggtaaaggcatgctaaccgctgcggtctgcggcgacgttttcgcctccccgagcgtggatgctgtactgaccgcgattcaggcggtgaccggtgaggctggctgtttgttgattgtgaaaaactacaccggtgaccgtcttaatttcggtctcgccgccgagaaggcgcgtcgccttggctataacgttgaaatgctgattgtcggcgacgacatctccctgccggataacaaacacccacgtggcattgcgggaactatcctggtgcataaaatcgcaggctattttgccgaacgcggctataacctcgccaccgtcctgcgtgaagcgcagtacgcagccagcaacacctttagcctgggcgtagcgctttccagctgtcatctgccgcaagaaaccgacgcagcccctcgtcatcatccgggtcatgcggagctgggtatgggaattcacggcgaaccaggcgcatcggttatcgacacccaaaacagtgcgcaagtggtaaacctgatggtggataaactgctggccgccctgcctgaaaccggtcgtctggcggtgatgattaataatcttggcggcgtttccgtggccgaaatggccatcatcacccgcgaactcgccagcagcccgctgcactcgcgtatcgactggctaattggcccggcctcgctggtcaccgcgctggatatgaaaggcttctcactgacggccatcgtgctggaagagagcatcgaaaaagcactgctcaccgaagtggaaaccagcaactggccgacgccggtcccaccgcgtgaaatcacctgcgtagtgtcatctcagcgtagcgcccgcgtggaattccagccttcggcaaacgccctggtggccgggattgtggagctggtcaccgcaaccctttccgatctggagactcatctgaatgcgctggacgccaaagtcggcgatggcgataccggttcgacctttgccgccggcgcgcgtgaaattgccagcctgctgcatcgccagcagctgccgctgaataaccttgccacgctgttcgcgctgattggcgaacgtctgaccgtggtgatgggcggttccagcggtgtgctgatgtcaatcttctttaccgccgccgggcagaaactggaacagggcgctaacgttgtcgaagcgctaaatacggggctggcgcagatgaagttctacggcggcgcagacgaaggcgatcgcacgatgattgatgcgctgcaaccggccctgacctcgctgctcgcacagccgaaaaatctgcaggccgcattcgacgccgcgcaagcgggagccgaacgaacctgtttgtcgagcaaagccaatgcgggtcgcgcatcgtatctgagcagcgaaagcctgctcggaaatatggaccccggcgcgcacgccgtagcgatggtgtttaaagcgctagcggagagtgagctgggctaa

SEQ ID NO: 28 - DNA, 11172 bp, Artificial
  DNA-Sequence of plasmid pDP_mgdf
tcgagtaggcctaagaaggagatatacatatgtacattatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaactcgagtgacgtcaagaaggagatatacatatggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgctctgctggtagccgaccagtacggtcagcgtttcctgcaagagtgggaataatttaaataagaaggagatatacatatgtctcaattcttttttaaccaacgcacccatcttgtgagcgacgtcatcgacggggcgattatcgccagcccatggaataacctggcgcgtctggaaagcgatccggccattcgcatcgtggtccgtcgtgaccttaataaaaataacgtagcggtcatttccggcggcggttcgggacacgaacccgcgcacgttgggtttatcggtaaaggcatgctaaccgctgcggtctgcggcgacgttttcgcctccccgagcgtggatgctgtactgaccgcgattcaggcggtgaccggtgaggctggctgtttgttgattgtgaaaaactacaccggtgaccgtcttaatttcggtctcgccgccgagaaggcgcgtcgccttggctataacgttgaaatgctgattgtcggcgacgacatctccctgccggataacaaacacccacgtggcattgcgggaactatcctggtgcataaaatcgcaggctattttgccgaacgcggctataacctcgccaccgtcctgcgtgaagcgcagtacgcagccagcaacacctttagcctgggcgtagcgctttccagctgtcatctgccgcaagaaaccgacgcagcccctcgtcatcatccgggtcatgcggagctgggtatgggaattcacggcgaaccaggcgcatcggttatcgacacccaaaacagtgcgcaagtggtaaacctgatggtggataaactgctggccgccctgcctgaaaccggtcgtctggcggtgatgattaataatcttggcggcgtttccgtggccgaaatggccatcatcacccgcgaactcgccagcagcccgctgcactcgcgtatcgactggctaattggcccggcctcgctggtcaccgcgctggatatgaaaggcttctcactgacggccatcgtgctggaagagagcatcgaaaaagcactgctcaccgaagtggaaaccagcaactggccgacgccggtcccaccgcgtgaaatcacctgcgtagtgtcatctcagcgtagcgcccgcgtggaattccagccttcggcaaacgccctggtggccgggattgtggagctggtcaccgcaaccctttccgatctggagactcatctgaatgcgctggacgccaaagtcggcgatggcgataccggttcgacctttgccgccggcgcgcgtgaaattgccagcctgctgcatcgccagcagctgccgctgaataaccttgccacgctgttcgcgctgattggcgaacgtctgaccgtggtgatgggcggttccagcggtgtgctgatgtcaatcttctttaccgccgccgggcagaaactggaacagggcgctaacgttgtcgaagcgctaaatacggggctggcgcagatgaagttctacggcggcgcagacgaaggcgatcgcacgatgattgatgcgctgcaaccggccctgacctcgctgctcgcacagccgaaaaatctgcaggccgcattcgacgccgcgcaagcgggagccgaacgaacctgtttgtcgagcaaagccaatgcgggtcgcgcatcgtatctgagcagcgaaagcctgctcggaaatatggaccccggcgcgcacgccgtagcgatggtgtttaaagcgctagcggagagtgagctgggctaaggcgcgccttataacctaggaagaaggagatatacatatgatggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaagcccgggcattaattaacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcatagttattaattaacacgtgaggccggccgagcggccgccaccgcggtggaggggcattaattgctagagggtcgaaattcaaattgtgagcggataactatttgaattttctgtatgaggttttgctaaacaactttcaacagtttcagcggagtgagaatagaaaggaacaactaaaggaattgcgaataataattttttcacgttgaaaatctccaaaaaaaaaggctccaaaaggagcctttaattgtatcggtttatcagcttgctttcgaggtgaatttcgaccctctagaggtcgaaattcaaattgtgagcggataacaatttgaattttctgtatgaggttttgctaaacaactttcaacagtttcagtggagtgagaatagaaaggaacaactaaaggaattgcgaataataattttttcacgttgaaaatctccaaaaaaaaaggctccaaaaggagcctttaattgtatcggtttatcagcttgctttcgaggtgaattttgaccctctagcgaaaatgcaagagcaaagacgaaaacatgccacacatgaggaataccgattctctcattaacatattcaggccagttatctgggcttaaaagcagaagtccaacccagataacgatcatatacatggttctctccagaggttcattactgaacactcgtccgagaataacgagtggatcccctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggacaccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgagaccttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctggcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtccatgggcaaatattatacgcaaggcgacaaggtgctgatgccgctggcgattcaggttcatcatgccgtttgtgatggcttccatgtcggcagaatgcttaatgaattacaacagtttttatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttggtacccaagtcgaccctgcagggtttaaaccagtattcaggtagctgttgagcctggggcggtagcgtgc

SEQ ID NO: 29 - protein, 367 aa, Escherichia coli
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 30 - protein, 552 aa, Escherichia coli
MSQFFFNQRTHLVSDVIDGAIIASPWNNLARLESDPAIRIVVRRDLNKNNVAVISGGGSGHEPAHVGFIGKGMLTAAVCGDVFASPSVDAVLTAIQAVTGEAGCLLIVKNYTGDRLNFGLAAEKARRLGYNVEMLIVGDDISLPDNKHPRGIAGTILVHKIAGYFAERGYNLATVLREAQYAASNTFSLGVALSSCHLPQETDAAPRHHPGHAELGMGIHGEPGASVIDTQNSAQVVNLMVDKLLAALPETGRLAVMINNLGGVSVAEMAIITRELASSPLHSRIDWLIGPASLVTALDMKGFSLTAIVLEESIEKALLTEVETSNWPTPVPPREITCVVSSQRSARVEFQPSANALVAGIVELVTATLSDLETHLNALDAKVGDGDTGSTFAAGAREIASLLHRQQLPLNNLATLFALIGERLTVVMGGSSGVLMSIFFTAAGQKLEQGANVVEALNTGLAQMKFYGGADEGDRTMIDALQPALTSLLAQPKNLQAAFDAAQAGAERTCLSSKANAGRASYLSSESLLGNMDPGAHAVAMVFKALAESELG

SEQ ID NO: 31 - protein, 383 aa, Escherichia coli
MMANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAW

SEQ ID NO: 32 - protein, 152 aa, Escherichia coli [sequence omitted]

SEQ ID NO: 33 - protein, 275 aa, Escherichia coli
MANPTVIKLQDGNVMPQLGLGVWQASNEEVITAIQKALEVGYRSIDTAAAYKNEEGVGKALKNASVNREELFITTKLWNDDHKRPREALLDSLKKLQLDYIDLYLMHWPVPAIDHYVEAWKGMIELQKEGLIKSIGVCNFQIHHLQRLIDETGVTPVINQIELHPLMQQRQLHAWNATHKIQTESWSPLAQGGKGVFDQKVIRDLADKYGKTPAQIVIRWHLDSGLVVIPKSVTPSRIAENFDVWDFRLDKDELGEIAKLDQGKRLGPDPDQFGG

SEQ ID NO: 34 - protein, 267 aa, Escherichia coli
MAIPAFGLGTFRLKDDVVISSVITALELGYRAIDTAQIYDNEAAVGQAIAESGVPRHELYITTKIWIENLSKDKLIPSLKESLQKLRTDYVDLTLIHWPSPNDEVSVEEFMQALLEAKKQGLTREIGISNFTIPLMEKAIAAVGAENIATNQIELSPYLQNRKVVAWAKQHGIHITSYMTLAYGKALKDEVIARIAAKHNATPAQVILAWAMGEGYSVIPSSTKRKNLESNLKAQNLQLDAEDKKAIAALDCNDRLVSPEGLAPEWD